(19) United States
(12) Patent Application Publication
(10) Pub. No.: US 2026/0358356 A1
(43) Pub. Date: Nov. 12, 2026

(54) METHOD FOR PREDICTION OF FUTURE HbA1c PARAMETER VALUE BASED ON CURRENT HbA1c VALUE AND STATUS OF TOTAL PLASMA N-GLYCOME

(71) Applicant: MEDILAB ONE d.o.o., Zagreb (HR)

(72) Inventors: Martiana DZEPINA, Zagreb (HR); Olga GORNIK, Zagreb (HR)

(73) Assignee: MEDILAB ONE d.o.o., Zagreb (HR)

(21) Appl. No.: 19/467,803

(22) Filed: Feb. 3, 2026

Related U.S. Application Data
(63) Continuation of application No. PCT/EP2023/072215, filed on Aug. 10, 2023.

(51) Int. Cl. G01N 33/72; G01N 33/58

(52) U.S. Cl. CPC G01N 33/723; G01N 33/582; G01N 2800/042

(57) ABSTRACT

The present disclosure reveals a process for the determination of the value of glycated hemoglobin (HbA1c) parameter in the future from current HbA1c value (cHbA1c) and quantitative analysis of thirteen specific total plasma protein N-glycans isolated from a blood sample of examined human subjects. Said combination of actual HbA1c value and 13 specific glycans were found to be significant diagnostic markers by statistical analysis of the results obtained from previously performed clinical trials that revealed the variation of quantitative glycans content in the blood plasma in a cohort of normal subjects, versus, those who experienced prediabetes or diabetes. From this analysis, new numerical models were developed which enable the calculation of the HbA1c (12 months) and HbA1c (24 months) parameters for estimation of HbA1c parameter values in the future, which provide an assessment of the eventual prediabetes or diabetes development in the examined subject in the future.

### 1. CROSS-REFERENCE TO RELATED APPLICATION

The present application is a continuation of International Application No. PCT/EP2023/072215, filed on Aug. 10, 2023, the entire disclosure of which is incorporated herein by reference.

### 2. TECHNICAL FIELD

The present disclosure relates to a method of predicting future glycated hemoglobin (HbA1c) parameter value as an assessment test for glycemic control, based on quantitative analysis of total plasma protein N-glycans and current HbA1c (cHbA1c) parameter value from a sample of the blood from the examined subject.

### 3. BACKGROUND

Glycans are complex carbohydrates composed of different monosaccharides, primarily N-acetyl-glucosamine (▪), fucose (▾), mannose (•), galactose (∘), and sialic acid (♦). Glycans are covalently bound to proteins, usually via an N-glycoside bond, and are involved in a multitude of physiological and pathological processes. Due to their influence on a large number of biological processes, they are recognized as important biochemical markers of overall health as well as various physiological and pathological conditions of the human body, see literature reference 1:
1) G. Opdenakker, P. M. Rudd, C. P. Ponting, R. A. Dwek: Concepts and principles of glycobiology, FASEB J. 7 (1993) 1330-1337.

Immunoglobulin G (IgG) is the most prevalent antibody in human plasma, playing an important role in defending the body against various pathogens. IgG is a glycoprotein, and the glycans attached to its heavy chains are particularly important for its stability and function.

The glycosylation of IgG is dependent on various physiological (age, sex, pregnancy) and pathological conditions (tumors, infections, autoimmune diseases). Changes in the pattern of IgG glycosylation during aging are known in the art, and by monitoring IgG N-glycans, it is possible to derive conclusions about the biological age of the subject being studied, see literature references 2-5:
2) R. Parekh, I. Roitt, D. Isenberg, R. Dwek, T. Rademacher: Age-related galactosylation of the N-linked oligosaccharides of human serum IgG, J. Exp. Med. 167 (1988) 1731-1736,
3) M. Pučić, A. Knežević, J. Vidič, B. Adamczyk, M. Novokmet, O. Polašek, O. Gornik, S. Šupraha-Goreta, M. R. Wormald, I. Redžić, H. Campbell, A. Wright, N. D. Hastie, J. F. Wilson, I. Rudan, M. Wuhrer, P. M. Rudd, D. Josić, G. Lauc: High Throughput Isolation and Glycosylation Analysis of IgG-Variability and Heritability of the IgG Glycome in Three Isolated Human Populations, Mol. Cell. Proteomics 10.10; doi:10.1074/mcp.M111.010090,
4) EP3011335B1; G. Lauc, M. Pučić-Baković, F. Vučković: Method for the analysis of N-glycans attached to immunoglobulin G from human blood plasma and its use; applicant: Genos d.o.o. (HR); priority date: 20.06.2013, and
5) J. Krištić, F. Vučković, C. Menni, L. Klarić, T. Keser, I. Beceheli, M. Pučić-Baković, M. Novokmet, M. Mangino, K. Thaqi, P. Rudan, N. Novokmet, J. Sarac, S. Missoni, I. Kolčić, O. Polašek, I. Rudan, H. Campbell, C. Hayward, Y. Aulchenko, A. Valdes, J. F. Wilson, O. Gornik, D. Primorac, V. Zoldoš, T. Spector, G. Lauc: Glycans are a novel biomarker of chronological and biological ages, J. Gerontol. A Biol. Sci. Med. Sci. 69 (2014) 779-789. doi: 10.1093/gerona/glt190.

Said posttranslational modifications of IgG are also connected with the development of various diseases including chronic inflammation, cancers, cardiovascular disease (CVD), and diabetes (T2D), among others, see literature references 6-12:
6) I. Gudelj, G. Lauc: Protein N-Glycosylation in Cardiovascular Disease and Related Risk Factors, Curr. Cardiovasc. Risk Rep. (2018) 12:16, https://doi.org/10.1007/s12170-018-0579-4
7) A. Birukov, B. Plavša, F. Eichelmann, O. Kuxhaus, R. A. Hoshi, N. Rudman, T. Štambuk, I. Trbojević-Akmačić, C. Schiborn, J. Morze, M. Mihelčić, A. Cindrić, Y. Liu, O. Demler, M. Perola, S. Mora, M. B. Schulze, G. Lauc, C. Wittenbecher: Immunoglobulin G N-Glycosylation Signatures in Incident Type 2 Diabetes and Cardiovascular Disease, Diabetes Care 45 (2022) 2729-2736,
8) S. Shkunnikova, A. Mijakovac, L. Sironic, M. Hanic, G. Lauc, M. Martinic Kavur: IgG glycans in health and disease: Prediction, intervention, prognosis, and therapy, Biotechnol. Adv. 67 (2023) 108169,
9) S. S. Singh, R. Heijmans, C. K. E. Meulen, A. G. Lieverse, O. Gornik, E. J. G. Sijbrands, G. Lauc, M. von Hoek: Association of the IgG N-glycome with the course of kidney function in type 2 diabetes, BMJ Open Diab. Res. Care 8 (2020) e001026,
10) E. Memarian, R. Heijmans, R. C. Slieker, A. Sierra, O. Gornik, J. W. J. Beulens, M. Hanic, P. Elders, J. Pascual, E. Sijbrands, G. Lauc, V. Dotz, C. Barrios, L. M. 't Hart, M. Wuhrer, M. van Hoek: IgG N-glycans are associated with prevalent and incident complications of type 2 diabetes, Diabetes Metab. Res. Rev. (2023) e3685,
11) M. Nemčić, M. Tijardović, N. Rudan, T. Bulum, M. Tomić, B. Plavša, S. Vučković Rebrina, M. Vučić Lovrenčić, L. Duvnjak, G. Morahan, O. Gornik: N-glycosylation of serum proteins in adult type 1 diabetes mellitus exposes further changes compared to children at the disease onset, Clin. Chim. Acta 543 (2023) 117298, and
12) E. Adua, E. Afrifa-Yamoah, E. Peprah-Yamoah, E. Odame Anto, E. Acheampong, K. A. Awuah-Mensah, W. Wang: Multi-block data integration analysis for identifying and validating targeted N-glycans as biomarkers for type II diabetes mellitus, Sci. Rep. 12 (2022) 10974.

Besides IgG, other plasma proteins undergo glycosylation yielding the corresponding N-glycans. The respective parameter of total plasma protein N-glycome is also an important marker for the development of various diseases, including insulin resistance (IR), T2D, inflammatory bowel disease (IBD), and colorectal cancer, among others, see literature reference 13-15:
13) V. Dotz, M. Wuhrer: N-glycome signatures in human plasma: associations with physiology and major diseases, FEBS Lett. 593 (2019) 2966-2976,
14) A. Cvetko, M. Mangino, M. Tijardović, D. Kifer, M. Falchi, T. Keser, M. Perola, T. D. Spector, G. Lauc, C. Menni, O. Gornik: Plasma N-glycome shows continuous deterioration as the diagnosis of insulin resistance approaches, BMJ Open Diab. Res. Care 9 (2021) e002263, and
15) P. Louca, T. Štambuk, A. Frkatović-Hodžić, A. Nogal, M. Mangino, S. E. Berry, H. Deriš, G. Hadjigeorgiou, J. Wolf, M. Vinicki, P. W. Franks, A. M. Valdes, T. D. Spector, G. Lauc, C. Menni: Plasma protein N-glycome composition associates with postprandial lipaemic response, BMC Med. 21 (2023) 231.

Additionally, glycated hemoglobin A1c (HbA1c) level is a well-known and standard parameter for measuring the amount of glucose bound to hemoglobin. It is a measure of the efficacy of the body's control of blood glucose levels. A value lower than 5.6 means that the examined subject is healthy concerning the glycemic status. A value between 5.7-6.4 means that the examined subject is in the condition of prediabetes, while a value equal to or higher than 6.5 means that the examined subject has developed diabetes, see literature references 16-18:
16) Executive Summary: Standards of Medical Care in Diabetes-2010, Diabetes Care 33 (2010) S4-S10,
17) T. Sitasuwan, R. Lertwattanarak: Prediction of type 2 diabetes mellitus using fasting plasma glucose and HbA1c levels among individuals with impaired fasting plasma glucose: a cross-sectional study in Thailand, BMJ Open 10 (2020) e041269, and
18) P. Vijayakumar, R. G. Nelson, R. L. Hanson, W. C. Knowler, M. Sinha: HbA1c and the Prediction of Type 2 Diabetes in Children and Adults, Diabetes Care 40 (2017) 16-21.

Moreover, the parameter of HbA1c levels has been already used as a key marker for the development of 5-years risk prediction model for type 2 diabetes in individuals with incident HbA1c-defined pre-diabetes, see literature reference 19:
19) S. K. Nicolaisen, R. W. Thomsen, C. J. Lau, H. T. Sørensen, L. Pedersen: Development of a 5-year risk prediction model for type 2 diabetes in individuals with incident HbA1c-defined pre-diabetes in Denmark, BMJ Open Diab. Res. Care 10 (2022) e002946.

The present disclosure solves the technical problem of the assessment of HbA1c value in the future in human subjects. This technical problem is solved in the present disclosure by the use of quantitative analysis of thirteen specific total plasma protein N-glycans as well as from current HbA1c (cHbA1c) value from one or more blood analyses of the examined subject. Said thirteen specific total plasma protein N-glycans, in combination with cHbA1c, proved to be specific markers for the prediction of future HbA1c levels.

### SUMMARY

The present disclosure reveals a method for the prediction of future glycated hemoglobin (HbA1c) parameter value in a human subject comprises of performing an analysis process of N-glycans of general formulae Ia-If, bound to plasma proteins,

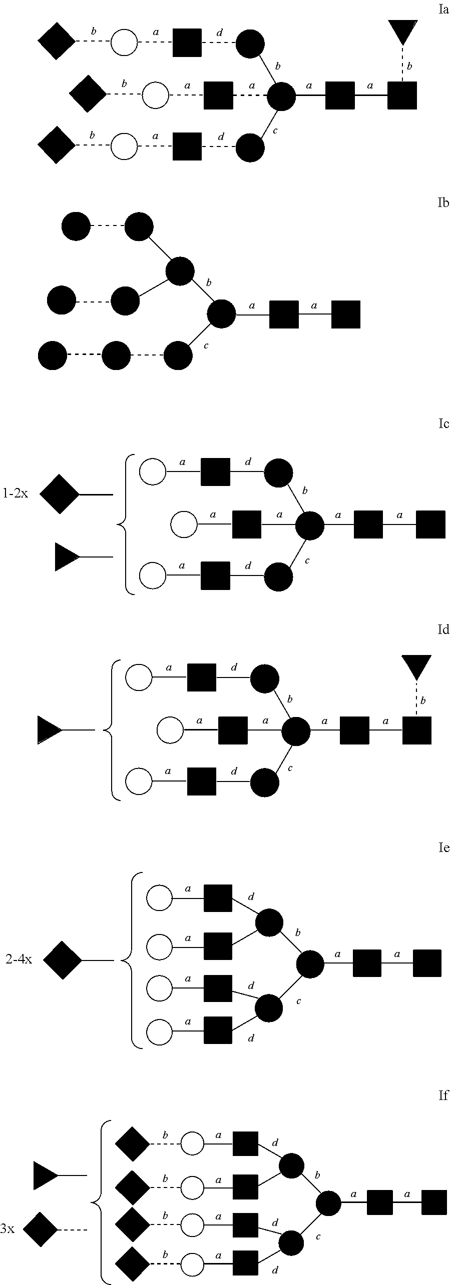

where symbols in Ia-If denote monomeric sugar units below:
▪ N-acetylglucosamine
▾ fucose
● mannose
◯ galactose
♦ sialic acid
   and where letters a-d in Ia-If determine types of glycoside bonds of said N-glycans Ia-If:
a=β(1-4)
b=α(1-6)
c=α(1-3)
d=β(1-2)

The said method comprises the following steps:
a) isolation of plasma from one or more blood samples that have been collected from the human subject under examination,
b) denaturation and release of said glycans plasma from total proteins by deglycosylation,
c) fluorescent derivatization with 2-aminobenzamide (2AB) and a reducing agent for reductive amination, optionally using a complex of picoline borane (BH₃·NC₅H₄-2-CH₃) or sodium cyanoborohydride (NaBH₃CN):

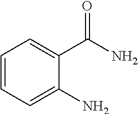

d) quantitative analysis of thus derivatized glycans by ultra-performance liquid chromatography (UPLC), which furnishes a thirty-nine separated glycan peaks GP1 . . . GP39, wherein numerical values of relative areas under respective glycan peaks GP3, GP5, GP6, GP10, GP14, GP17, GP18, GP19, GP22, GP29, GP32, GP33, and GP36 within the corresponding UPLC chromatogram are obtained as the numerical values {GP3, GP5, GP6, GP10, GP14, GP17, GP18, GP19, GP22, GP29, GP32, GP33, and GP36}, which corresponds to the glycan structures given in Table below:

| | Glycan nomenclature/ | | |
|---|---|---|---|
| No. | composition | Code | Structure |
| 1 | A2 [6] BG1/H4N5 | GP3 | [symbol] |
| 2 | FA2 [3] G1/H4N4F1 | GP5 | [symbol] |
| 3 | FA2 [6] BG1/H4N5F1 | GP6 | [symbol] |
| 4 | FA2G2/H5N4F1 | GP10 | [symbol] |
| 5 | A2G2S1/H5N4S1 | GP14 | [symbol] |
| 6 | FA2BG2S1/H5N5F1S1 | GP17 | [symbol] |
| 7 | A2G2S2/H5N4S2 | GP18 | [symbol] |
| | FA2G2S2/H5N4F1S2 | | [symbol] |
| 8 | M9/H9N2 | GP19 | [symbol] |
| 9 | FA2G2S2/H5N4F1S2 | GP22 | [symbol] |
| 10 | A3G3S3/H6N5S3 | GP29 | [symbol] |
| | A3F1G3S2/H6N5F1S2 | | [symbol] |
| 11 | A3G3S3/H6N5S3 | GP32 | [symbol] |
| | A4G4S2/H7N6S2 | | [symbol] |
| 12 | A3F1G3S3/H6N5F1S3 | GP33 | [symbol] |
| 13 | A4G4S3/H7N6S3 | GP36 | [symbol] |
| | A4F1G4S3/H7N6F1S3 | | [symbol] |

e) as well as analysis of the current HbA1c parameter value (cHbA1c) from the same blood samples that have been collected from the human subject under examination,
f) where numerical results of relative areas under the selected glycan peaks GP3, GP5, GP6, GP10, GP14, GP17, GP18, GP19, GP22, GP29, GP32, GP33, and GP36, that correspond to values {GP3, GP5, GP6, GP10, GP14, GP17, GP18, GP19, GP22, GP29, GP32, GP33, and GP36} calculated in step d, and numerical result of the CHbA1c parameter value obtained in step e, are included in a model for calculation of the predictive value of the HbA1c parameter in the future, which is a function of the thirteen glycan arguments and CHbA1c parameter value:

HbA1c(T)=HbA1c((GP3,GP5,GP6,GP10,GP14,GP17,GP18,GP19,GP22,GP29,GP32,GP33,GP36),cHbA1c)
where HbA1c (T) parameter means HbA1c value in time point T, having a value of 12 or 24 months in the future, and
g) determination of said predictive, future HbA1c parameter value, HbA1c(12 months) and/or HbA1c(24 months) and linking of the said parameter with the probability that the examined subject will develop prediabetes, diabetes, or remain healthy after the said period of 12 or 24 months.

Additionally, the method according to the present disclosure further comprising: obtaining the future HbA1c parameter value model via statistical data analysis performed after a prospective study that determines the variation of quantitative total plasma protein N-glycans {GP1, . . . , GP39} content and current HbA1c (cHbA1c) parameter value in the blood plasma in the following sub-groups of participants included in the study:
(i) those that had diabetes at the 0-month time point and remained in the diabetes condition after 12 months,
(ii) those that had diabetes at the 0-month time point but have become healthy after 12 months,
(iii) those that had diabetes at the 0-month time point but have developed prediabetes after 12 months,
(iv) those that were healthy at the 0-month time point but have developed diabetes after 12 months,
(v) those that were healthy at the 0-month time point and remained healthy after 12 months,
(vi) those that were healthy at the 0-month time point but have developed prediabetes after 12 months,
(vii) those that had prediabetes at the 0-month time point but have developed diabetes after 12 months,
(viii) those that had prediabetes at the 0-month time point and have become healthy after 12 months, and
(ix) those that had prediabetes at the 0-month time point and remained in the prediabetes condition after 12 months.

The set of predictive total plasma protein N-glycans is determined using regression models that are corrected for multiple confounders, including age, blood glucose concentration, HbA1c parameter value, weight, height, waist, body mass index (BMI), from which total plasma protein N-glycome and cHbA1c parameter value-based predictive model is constructed based on the determined total plasma protein N-glycans and cHbA1c value construct.

The method according to the present disclosure reveals the numerical prediction model for future HbA1c value by the formula given below:

$$HbA 1 c ( 12 months ) = 0.6695 + 0.7857 \cdot HbA 1 c ( 0 ) + 0.0362 \cdot logit ( GP 3 ) ++ ( - 0.0897 \cdot logit ( GP 14 ) ) + ( - 0.0614 \cdot logit ( GP 17 ) ) ++ ( - 0.0596 \cdot logit ( GP 18 ) ) + ( 0.0934 \cdot logit ( GP 19 ) ) ++ ( - 0.0339 \cdot logit ( GP 29 ) ) + ( - 0.0651 \cdot logit ( GP 36 ) )$$

where:
HbA1c(12 months) is the predicted HbA1c value for the examined subject for 12 months in the future,
HbA1c(0) is the current HbA1c value (cHbA1c) of the examined subject at the time of examination, and
the calculated value for HbA1c(12 months) reveals the following conclusion for the examined subject:
A. HbA1c(12 months) is <5.7, then the examined subject will be healthy,
B. HbA1c(12 months) is 5.7-6.7, then the examined subject will develop prediabetes, with the prediction probability at the level of 78%, and 22% probability that will be healthy, and
c. HbA1c(12 months) is >6.7, then the examined subject will develop diabetes, with the prediction probability at the level of 78%, and 22% probability that will develop prediabetes.

In another embodiment of this invention, the method involves the prediction of future HbA1c parameter value prediction model as given with the formula below:

$$HbA 1 c ( 24 months ) = 1.0314 \cdot Intercept + 0.6569 \cdot HbA 1 c ( prop . before ) ++ ( - 0.1167 \cdot logit ( GP 10 ) ) + ( - 0.0717 \cdot logit ( GP 18 ) ) ++ ( 0.1109 \cdot logit ( GP 32 ) ) + ( - 0.0578 \cdot logit ( GP 33 ) ) ++ ( - 0.1456 \cdot logit ( GP 36 ) ) + ( 0.2648 \cdot logit ( GP5\_ 12 -- GP5\_ 0 ) ) + ( - 0.4114 \cdot logit ( GP6\_ 12 - GP6\_ 0 ) ) ++ ( 0.0963 \cdot logit ( GP22\_ 12 - GP22\_ 0 ) ) ++ ( - 0.0885 \cdot logit ( GP33\_ 12 - GP33\_ 0 )$$

where:
Intercept is a value on the y-axis when all factors in the said model are equal to zero,
HbA1c(prop.before) is a proportion of glycated HbA1c expressed in percentage (%) to total HbA1c,
GPX_Y is a value of the result of the corresponding glycan peak GP of number X, GPX, determined in the respective time point expressed in months, Y, which can be: Y=0 or Y=12 months, and
the calculated value for HbA1c(24 months) reveals the following conclusion for the examined subject:
   A. HbA1c(24 months) is <5.7, then the examined subject will be healthy,
   B. HbA1c(24 months) is 5.7-6.7, then the examined subject will develop prediabetes, with the prediction probability at the level of 70%, and 30% probability that will be healthy, and
   C. HbA1c(24 months) is >6.7, then the examined subject will develop diabetes, with the prediction probability at the level of 70%, and 30% probability that will develop prediabetes.

In a further embodiment of this disclosure, the determination of the total plasma protein N-glycans under the peaks GP3, GP5, GP6, GP10, GP14, GP17, GP18, GP19, GP22, GP29, GP32, GP33, and GP36 from the chromatogram in FIG. 1 can be performed by alternative quantitative analysis techniques selected from the group consisting of: MALDI-TOF mass spectrometry, liquid chromatography coupled with mass spectrometry (LC-MS), or capillary electrophoresis (CE), or by LC-MS analysis of the corresponding glycopeptides.

According to the present invention, the diagnostic process is used for predicting the future value of the HbA1c parameter in human subjects that is very likely to occur within 12 or 24 months.

### BRIEF DESCRIPTION OF DRAWINGS

In order to explain the technical features of embodiments of the present disclosure more clearly, the drawings used in the present disclosure are briefly introduced as follows. Obviously, the drawings in the following description are some exemplary embodiments of the present disclosure. Ordinary persons skilled in the art may obtain other drawings and features based on these disclosed drawings without inventive efforts.

FIG. 1. A typical chromatogram of 2-amino-benzamide (2AB) derived total plasma protein N-glycans obtained by the ultra-high performance liquid chromatography (HILIC-UPLC)-based method described in Example 1, with 39 separated chromatographic peaks which are further in the text designed as glycan peaks GP1-GP39.

FIGS. 2-21. Flowcharts with the results of variation of blood glucose, HbA1c, weight, height, waist, body mass index GP6, GP10, (BMI), and GP3, GP5, GP14, GP17, GP18, GP19, GP22, GP29, GP32, GP33, and GP36 in subjects under the study of the dependence of the concentration of total plasma protein N-glycans GP1-GP39 with the glycemic status according to the concentration of glycated hemoglobin (HbA1c) parameter value, in the text also expressed as a current HbA1c (cHbA1c), in different participant groups at starting point of the study (0-month), at 12 months point, and the difference between 12 months value and initial value (0-month). The groups are classified according to the observed transition:
(1) those that had diabetes at the 0-month time point and remained in the diabetes condition after 12 months (□; Diabetes->Diabetes);
(2) those that had diabetes at the 0-month time point but have developed prediabetes after 12 months (A; Diabetes->Prediabetes);
(3) those that were healthy at the 0-month time point but have developed diabetes after 12 months (+; No->Diabetes).
(4) those that were healthy at the 0-month time point and remained healthy after 12 months (x; No->No);
(5) those that were healthy at the 0-month time point but have developed prediabetes after 12 months (0); No->Prediabetes);
(6) those that had prediabetes at the 0-month time point but have developed diabetes after 12 months (▪; Prediabetes->Diabetes)
(7) those that had prediabetes at the 0-month time point and have become healthy after 12 months (♦; Prediabetes->No); and
(8) those that had prediabetes at the 0-month time point and remained in the prediabetes condition after 12 months (•; Prediabetes->Prediabetes).
Error bars are 95% CI. A detailed description of the study is given in Example 2.

FIG. 2. A flowchart with the results of variation of blood glucose concentration [mmol/L] in the study, see Example 2.

FIG. 3. A flowchart with the results of variation of HbA1c [%] in the study, see Example 2.

FIG. 4. A flowchart with the results of variation of HbA1c [mmol/mol] in the study, see Example 2.

FIG. 5. A flowchart with the results of variation of body weight [kg] of the subjects involved in the study, see Example 2.

FIG. 6. A flowchart with the results of variation of body height [cm] of the subjects involved in the study, see Example 2.

FIG. 7. A flowchart with the results of variation of waist [cm] of the subjects involved in the study, see Example 2.

FIG. 8. A flowchart with the results of variation of body mass index (BMI) [kg/m²] of the subjects involved in the study, see Example 2.

FIG. 9. A flowchart with the results of variation of age [years] of the subjects involved in the study, see Example 2.

FIG. 10. A flowchart with the results of variation of GP3 in the study, see Example 2.

FIG. 11. A flowchart with the results of variation of GP5 in the study, see Example 2.

FIG. 12. A flowchart with the results of variation of GP6 in the study, see Example 2.

FIG. 13. A flowchart with the results of variation of GP10 in the study, see Example 2.

FIG. 14. A flowchart with the results of variation of GP14 in the study, see Example 2.

FIG. 15. A flowchart with the results of variation of GP17 in the study, see Example 2.

FIG. 16. A flowchart with the results of variation of GP18 in the study, see Example 2.

FIG. 17. A flowchart with the results of variation of GP19 in the study, see Example 2.

FIG. 18. A flowchart with the results of variation of GP22 in the study, see Example 2.

FIG. 19. A flowchart with the results of variation of GP29 in the study, see Example 2.

FIG. 20. A flowchart with the results of variation of GP32 in the study, see Example 2.

FIG. 21. A flowchart with the results of variation of GP33 in the study, see Example 2.

FIG. 22. A flowchart with the results of variation of GP36 in the study, see Example 2.

FIG. 23. A flowchart with the coincidence of HbA1c prediction at 12 months in the future based on current HbA1c parameter value (cHbA1c) and analyzed total plasma protein N-glycans GP3, GP14, GP17, GP18, GP19, GP29 and GP36. R²=57.0%. Correctly classified=78.38. See the study details described in Example 2.

FIG. 24. A flowchart with the coincidence of HbA1c prediction at 24 months in the future based on the change of HbA1c and total plasma protein N-glycans GP10, GP18, GP32, GP33, GP36, and differences of GP5, GP6, GP22, and GP33 values between 12-month results and 0-month results within the first 12 months. R²=50.5%. Correctly classified: 77.08. See the study details described in Example 2.

### DETAILED DESCRIPTION

The present disclosure reveals a method for the prediction of future glycated hemoglobin (HbA1c) parameter value in a human subject comprises of performing an analysis process of N-glycans of general formulae Ia-If, bound to plasma proteins,

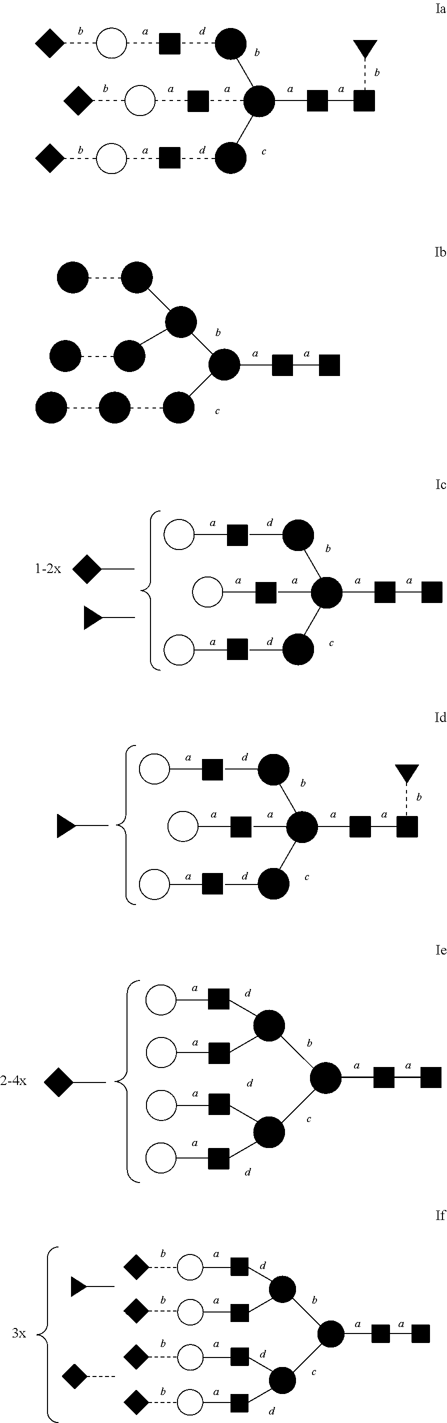

where symbols in Ia-If denote monomeric sugar units below:
   ▪ N-acetylglucosamine
   ▾ fucose
   ● mannose
   ◯ galactose
   ♦ sialic acid
and where letters a-d in Ia-If determine types of glycoside bonds of said N-glycans Ia-If:
   a=β(1-4)
   b=α(1-6)
   c=α(1-3)
   d=β(1-2)
and where the said method comprises the following steps:
a) isolation of plasma from one or more blood samples that have been collected from the human subject under examination,
b) denaturation and release of said glycans from total plasma proteins by deglycosylation,
c) fluorescent derivatization with 2-aminobenzamide (2AB) and a reducing agent for reductive amination, optionally using a complex of picoline borane (BH₃·NC₅H₄-2-CH₃) or sodium cyanoborohydride (NaBH₃CN):

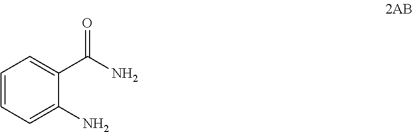

d) quantitative analysis of thus derivatized glycans by ultra-performance liquid chromatography (UPLC), which furnishes a thirty-nine separated glycan peaks GP1 . . . GP39, which corresponds to the glycan structures given in the Table below:

| | Glycan nomenclature/ | | |
|---|---|---|---|
| No. | composition | Code | Structure |
| 1 | FA2/H3N4F1 | GP1 | [symbol] |
| 2 | FA2B/H3N5F1 | GP2 | [symbol] |
| | M5/H5N2 | | [symbol] |
| | A2 [6] G1/H4N4 | | [symbol] |
| 3 | A2 [6] BG1/H4N5 | GP3 | [symbol] |
| 4 | FA2 [6] G1/H4N4F1 | GP4 | [symbol] |
| 5 | FA2 [3] G1/H4N4F1 | GP5 | [symbol] |
| 6 | FA2 [6] BG1/H4N5F1 | GP6 | [symbol] |
| 7 | M6/H6N2 | GP7 | [symbol] |
| | FA2 [3] BG1/H4N5F1 | | [symbol] |
| 8 | A2G2/H5N4 | GP8 | [symbol] |
| | A1G1S1/H4N3S1 | | [symbol] |
| 9 | A2BG2/H5N5 | GP9 | [symbol] |
| | FA2G2/H5N4F1 | | [symbol] |
| 10 | FA2G2/H5N4F1 | GP10 | [symbol] |
| 11 | FA2BG2/H5N5F1 | GP11 | [symbol] |
| | A2G1S1/H4N4S1 | | [symbol] |
| 12 | M7/H7N2 | GP12 | [symbol] |
| | A2G2S1/H5N4S1 | | [symbol] |
| | M4A1G1S1/H5N3S1 | | [symbol] |
| | A2BG2S1/H4N5S1 | | [symbol] |
| 13 | FA2G1S1/H4N4F1S1 | GP13 | [symbol] |
| | FA2BG1S1/H4N5F1S1 | | [symbol] |
| | A2G2S1/H5N4S1 | | [symbol] |
| 14 | A2G2S1/H5N4S1 | GP14 | [symbol] |
| 15 | A2BG2S1/H5N5S1 | GP15 | [symbol] |
| | A2G2S1/H5N4S1 | | [symbol] |
| 16 | FA2G2S1/H5N4F1S1 | GP16 | [symbol] |
| | M8/H8N2 | | [symbol] |
| 17 | FA2BG2S1/H5N5F1S1 | GP17 | [symbol] |
| 18 | A2G2S2/H5N4S2 | GP18 | [symbol] |
| | FA2G2S2/H5N4F1S2 | | [symbol] |
| 19 | M9/H9N2 | GP19 | [symbol] |
| 20 | A2G2S2/H5N4S2 | GP20 | [symbol] |
| 21 | A2G2S2/H5N4S2 | GP21 | [symbol] |
| | A3G3S1/H6N5S1 | | [symbol] |
| | A3F1G3S1/H6N5F1S1 | | [symbol] |
| | A2BG2S2/H6N5S2 | | [symbol] |
| 22 | FA2G2S2/H5N4F1S2 | GP22 | [symbol] |
| 23 | FA2BG2S2/H5N5F1S2 | GP23 | [symbol] |
| | FA2G2S2/H5N4F1S2 | | [symbol] |
| 24 | A3G3S2/H6N5S2 | GP24 | [symbol] |
| | A3F1G3S1/H6N5F1S1 | | [symbol] |
| 25 | A3G3S2/H6N5S2 | GP25 | [symbol] |
| | FA2F1G2S2/H5N4F2S2 | | [symbol] |
| | A3F1G3S2/H6N5F1S1 | | [symbol] |
| 26 | A3G3S2/H6N5S2 | GP26 | [symbol] |
| | A3F1G3S2/H6N5F1S1 | | [symbol] |
| 27 | A3F1G3S2/H6N5F1S1 | GP27 | [symbol] |
| 28 | A3F1G3S2/H6N5F1S2 | GP28 | [symbol] |
| | A3G3S3/H6N5S3 | | [symbol] |
| 29 | A3G3S3/H6N5S3 | GP29 | [symbol] |
| | A3F1G3S2/H6N5F1S2 | | [symbol] |
| 30 | A3G3S3/H6N5S3 | GP30 | [symbol] |
| | A4G4S2/H7N6S2 | | [symbol] |
| | A3F1G3S3/H6N5F1S3 | | [symbol] |
| 31 | FA3G3S3/H6N5F1S3 | GP31 | [symbol] |
| 32 | A3G3S3/H6N5S3 | GP32 | [symbol] |
| | A4G4S2/H7N6S2 | | [symbol] |
| 33 | A3F1G3S3/H6N5F1S3 | GP33 | [symbol] |
| 34 | FA3G3S3/H6N5F1S3 | GP34 | [symbol] |
| | A4G4S3/H7N6S3 | | [symbol] |
| 35 | FA3F1G3S3/H6N5F2S3 | GP35 | [symbol] |
| | A4G4S3/H7N6S3 | | [symbol] |
| 36 | A4G4S3/H7N6S3 | GP36 | [symbol] |
| | A4F1G4S3/H7N6F1S3 | | [symbol] |
| 37 | A4G4S4/H7N6S4 | GP37 | [symbol] |
| | A4F1G4S3/H7N6F1S3 | | [symbol] |
| 38 | A4G4S4/H7N6S4 | GP38 | [symbol] |
| | A4F1G4S3/H7N6F1S3 | | [symbol] |
| | A4F1G4S4/H7N6F1S4 | | [symbol] |
| 39 | A4F1G4S4/H7N6F1S4 | GP39 | [symbol] |

wherein numerical values of relative areas under respective glycan peaks GP3, GP5, GP6, GP10, GP14, GP17, GP18, GP19, GP22, GP29, GP32, GP33, and GP36 within the corresponding UPLC chromatogram are obtained as the numerical values {GP3, GP5, GP6, GP10, GP14, GP17, GP18, GP19, GP22, GP29, GP32, GP33, and GP36},
e) as well as analysis of the current HbA1c parameter value (cHbA1c) from the same blood samples that have been collected from the human subject under examination,
f) where numerical results of relative areas under the selected glycan peaks GP3, GP5, GP6, GP10, GP14, GP17, GP18, GP19, GP22, GP29, GP32, GP33, and GP36, that correspond to values {GP3, GP5, GP6, GP10, GP14, GP17, GP18, GP19, GP22, GP29, GP32, GP33, and GP36} calculated in step d, and numerical result of the cHbA1c parameter value obtained in step e, are included in a model for calculation of the predictive value of the HbA1c parameter in the future, which is a function of the thirteen glycan arguments and CHbA1c parameter value:

HbA1c(T)=HbA1c((GP3,GP5,GP6,GP10,GP14,GP17,GP18,GP19,GP22,GP29,GP32,GP33,GP36),cHbA1c)
where HbA1c (T) parameter means HbA1c value in time point T, having a value of 12 or 24 months in the future, and
g) determination of said predictive, future HbA1c parameter value, HbA1c(12 months) and/or HbA1c(24 months) and linking of the said parameter with the probability that the examined subject will develop prediabetes, diabetes, or remain healthy after the said period of 12 or 24 months.

Additionally, the method according to the present disclosure further comprising: obtaining the future HbA1c parameter value model via statistical data analysis performed after a prospective study that determines the variation of quantitative total plasma protein N-glycans {GP1, . . . , GP39} content and current HbA1c (cHbA1c) parameter value in the blood plasma in the following sub-groups of participants included in the study:
(i) those that had diabetes at the 0-month time point and remained in the diabetes condition after 12 months (a sub-group marked with: □; Diabetes->Diabetes; 1 participant),
(ii) those that had diabetes at the 0-month time point but have become healthy after 12 months (a sub-group marked with: Diabetes->No; 0 participants),
(iii) those that had diabetes at the 0-month time point but have developed prediabetes after 12 months (a sub-group marked with: Δ; Diabetes->Prediabetes; 0 participants),
(iv) those that were healthy at the 0-month time point but have developed diabetes after 12 months (a sub-group marked with: +; No->Diabetes; 6 participants),
(v) those that were healthy at the 0-month time point and remained healthy after 12 months (a sub-group marked with: x; No->No; 801 participants),
(vi) those that were healthy at the 0-month time point but have developed prediabetes after 12 months (a sub-group marked with: 0; No->Prediabetes; 113 participants),
(vii) those that had prediabetes at the 0-month time point but have developed diabetes after 12 months (a sub-group marked with: ▪; Prediabetes->Diabetes; 30 participants),
(viii) those that had prediabetes at the 0-month time point and have become healthy after 12 months (a sub-group marked with: ♦; Prediabetes->No; 282 participants), and
(ix) those that had prediabetes at the 0-month time point and remained in the prediabetes condition after 12 months (a sub-group marked with: •; Prediabetes->Prediabetes; 593 participants),
   wherein the set of predictive total plasma protein N-glycans are determined using regression models that are corrected for multiple confounders, including age, blood glucose concentration, HbA1c parameter value, weight, height, waist, body mass index (BMI), from which total plasma protein N-glycome and cHbA1c parameter value-based predictive model is constructed based on the determined total plasma protein N-glycans and cHbA1c value construct.

The typical process for performing the analysis of total plasma N-glycans, which includes steps a-d of the method according to the present disclosure, is described in Example 1.

Additionally, a typical method for the determination of the current HbA1c (cHbA1c) parameter value, which represents a step e of the method according to the present disclosure, is a standard clinical method, e.g., see literature reference 20 for one of the common analytical protocols:
20) Cobas®; Turbidimetric inhibition immunoassay (TINIA) for the in vitro determination of hemoglobin A1c in whole blood or hemolysate; Tina-quant Hemoblobin A1c Gen. 3—Whole blood application-Standardized according to IFCC transferable to DCCT/NGSP; available at the link below:
   https://diagnostics.roche.com/global/en/products/params/tina-quant-hba1c-gen-3.html

The raw data about the relationship between total plasma protein N-glycans and current HbA1c value, and the incidence of future development of prediabetes of diabetes, was obtained from the study performed on a large population of human subjects (N=1826 participants) that have diabetes indication in their family and were monitored at starting time point and after 12 months regarding their glycemic status against total plasma protein N-glycome and HbA1c value. The details of this study are described in Example 2.

From the results obtained in this study, thirteen key glycans GP3, GP5, GP6, GP10, GP14, GP17, GP18, GP19, GP22, GP29, GP32, GP33, and GP36 have been identified as key markers, together with current HbA1c (cHbA1c) parameter, as especially informative for the prediction of future HbA1c parameter value and are used for the generation of the numerical model according to the present invention.

The method according to the present disclosure reveals the numerical prediction model for future HbA1c value by the formula given below:

$$HbA 1 c ( 12 months ) = 0.6695 + 0.7857 \cdot HbA 1 c ( 0 ) + 0.0362 \cdot logit ( GP 3 ) ++ ( - 0.0897 \cdot logit ( GP 14 ) ) + ( - 0.0614 \cdot logit ( GP 17 ) ) ++ ( - 0.0596 \cdot logit ( GP 18 ) ) + ( 0.0934 \cdot logit ( GP 19 ) ) ++ ( - 0.0339 \cdot logit ( GP 29 ) ) + ( - 0.0651 \cdot logit ( GP 36 ) )$$

where:
HbA1c(12 months) is the predicted HbA1c value for the examined subject for 12 months in the future,
HbA1c(0) is the current HbA1c value (cHbA1c) of the examined subject at the time of examination, and
the calculated value for HbA1c(12 months) reveals the following conclusion for the examined subject:
A. HbA1c(12 months) is <5.7, then the examined subject will be healthy,
B. HbA1c(12 months) is 5.7-6.7, then the examined subject will develop prediabetes, with the prediction probability at the level of 78%, and 22% probability that will be healthy, and
C. HbA1c(12 months) is >6.7, then the examined subject will develop diabetes, with the prediction probability at the level of 78%, and 22% probability that will develop prediabetes.

In another embodiment of this invention, the method involves the prediction of future HbA1c parameter value prediction model as given with the formula below:

$$HbA 1 c ( 24 months ) = 1.0314 \cdot Intercept + 0.6569 \cdot HbA 1 c ( prop . before ) ++ ( - 0.1167 \cdot logit ( GP 10 ) ) + ( - 0.0717 \cdot logit ( GP 18 ) ) ++ ( 0.1109 \cdot logit ( GP 32 ) ) + ( - 0.0578 \cdot logit ( GP 33 ) ) ++ ( - 0.1456 \cdot logit ( GP 36 ) ) + ( 0.2648 \cdot logit ( GP5\_ 12 -- GP5\_ 0 ) ) + ( - 0.4114 \cdot logit ( GP6\_ 12 - GP6\_ 0 ) ) ++ ( 0.0963 \cdot logit ( GP22\_ 12 - GP22\_ 0 ) ) ++ ( - 0.0885 \cdot logit ( GP33\_ 12 - GP33\_ 0 )$$

where:
Intercept is a value on the y-axis when all factors in the said model are equal to zero,
HbA1c(prop.before) is a proportion of glycated HbA1c expressed in percentage (%) to total HbA1c,
GPX_Y is a value of the result of the corresponding glycan peak GP of number X, GPX, determined in the respective time point expressed in months, Y, which can be: Y=0 or Y=12 months, and
the calculated value for HbA1c(24 months) reveals the following conclusion for the examined subject:
A. HbA1c(24 months) is <5.7, then the examined subject will be healthy,
B. HbA1c(24 months) is 5.7-6.7, then the examined subject will develop prediabetes, with the prediction probability at the level of 70%, and 30% probability that will be healthy, and
C. Hba1c(24 months) is >6.7, then the examined subject will develop diabetes, with the prediction probability at the level of 70%, and 30% probability that will develop prediabetes.

The development of both numerical models according to the present invention as well as statistical procedures employed for their generation from the raw data obtained from the study performed in human subjects are described in Example 2.

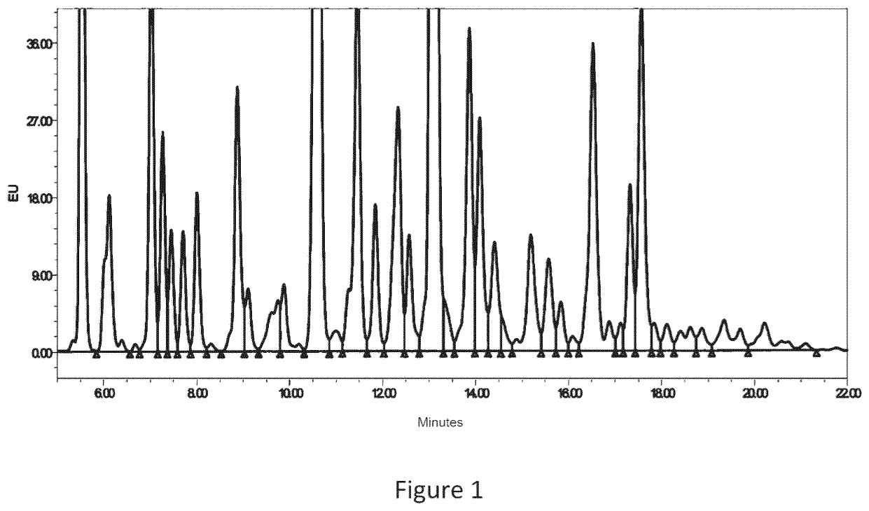

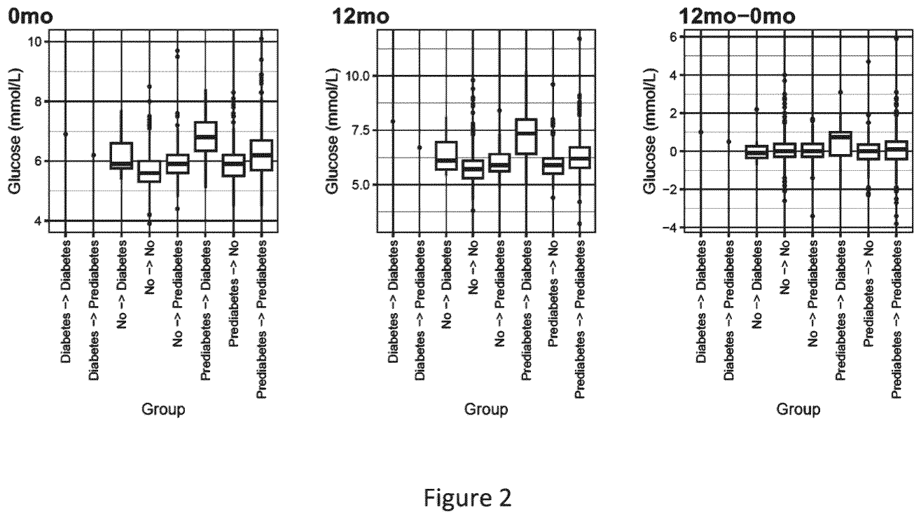

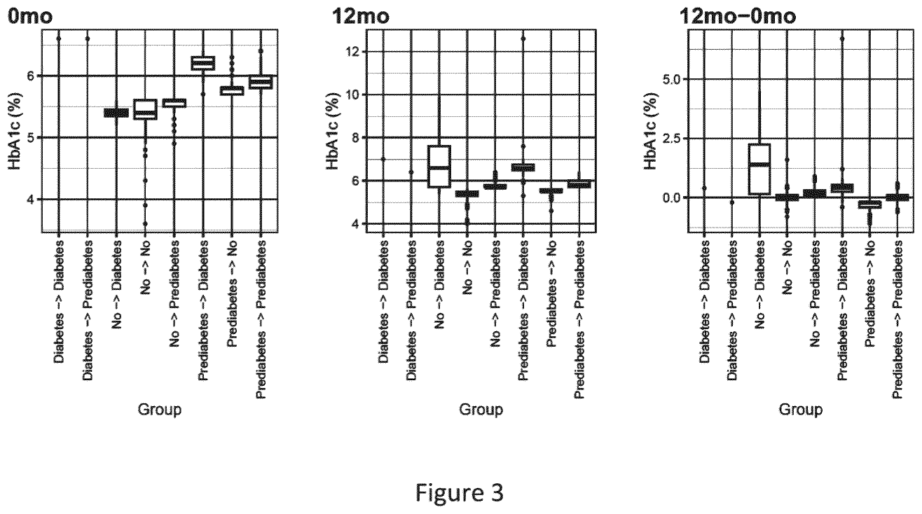

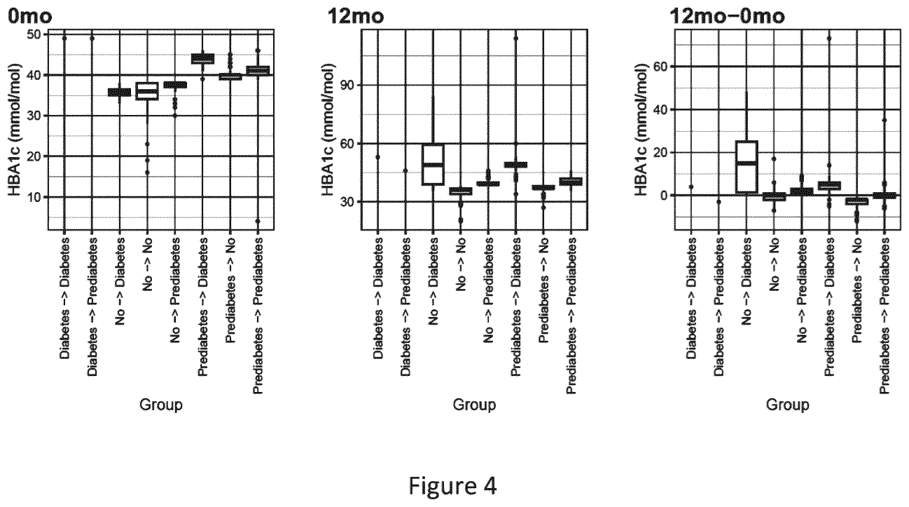

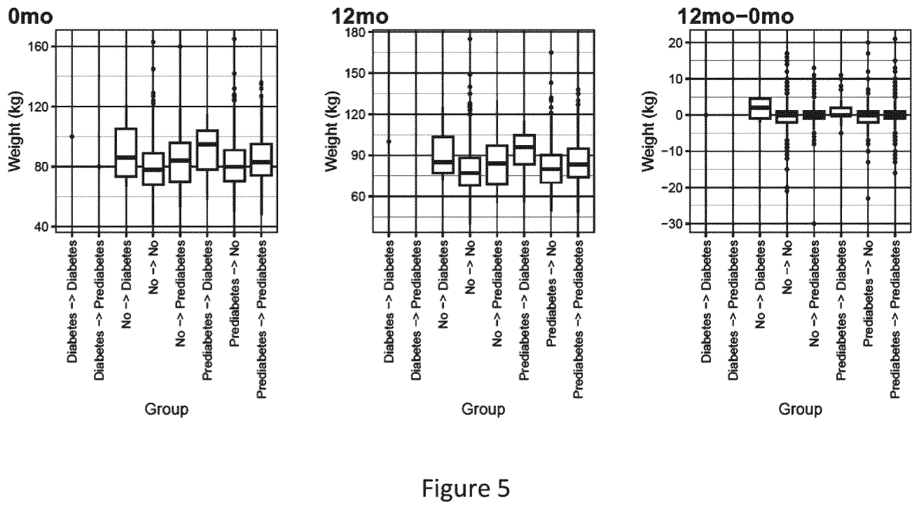

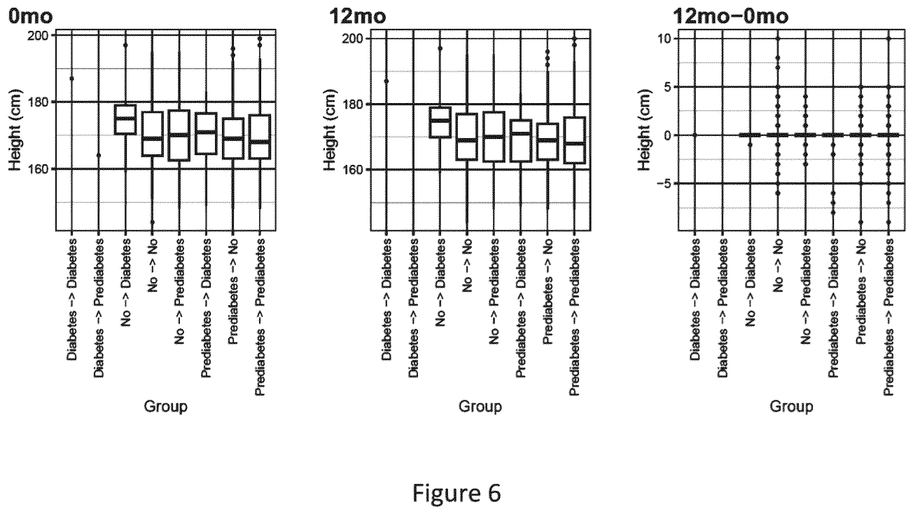

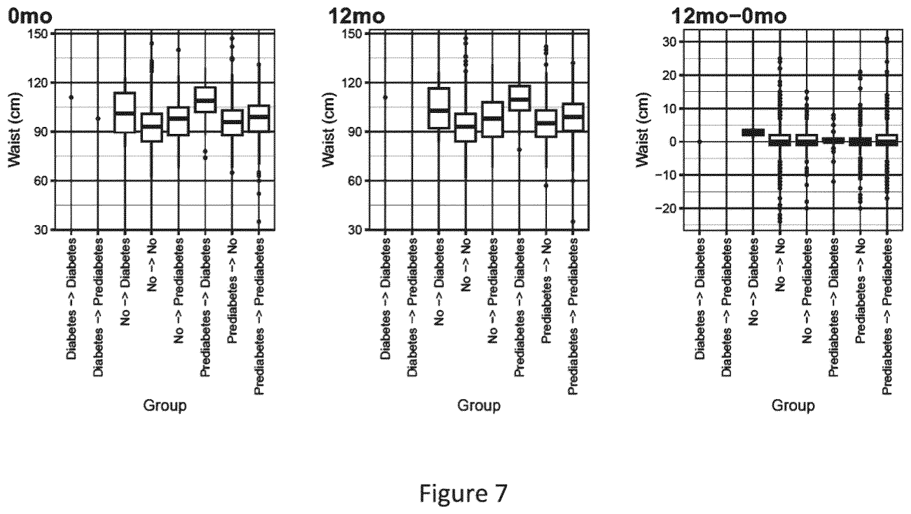

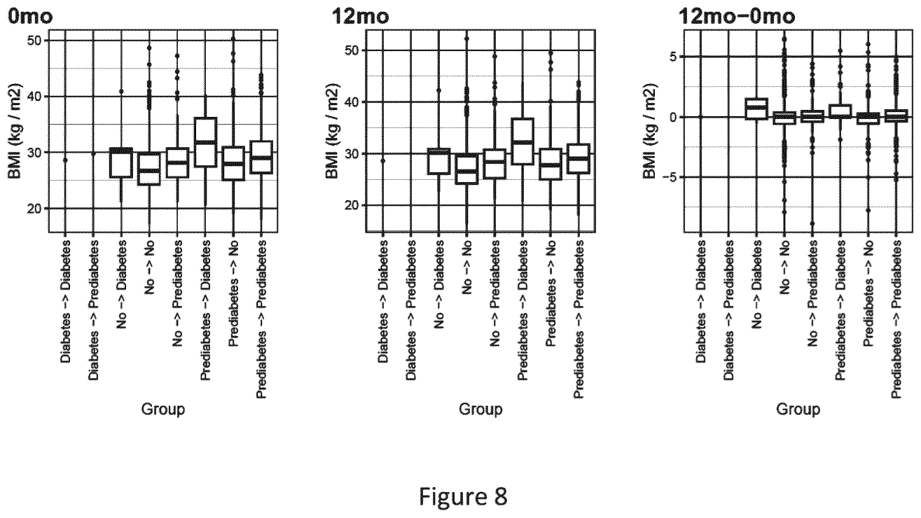

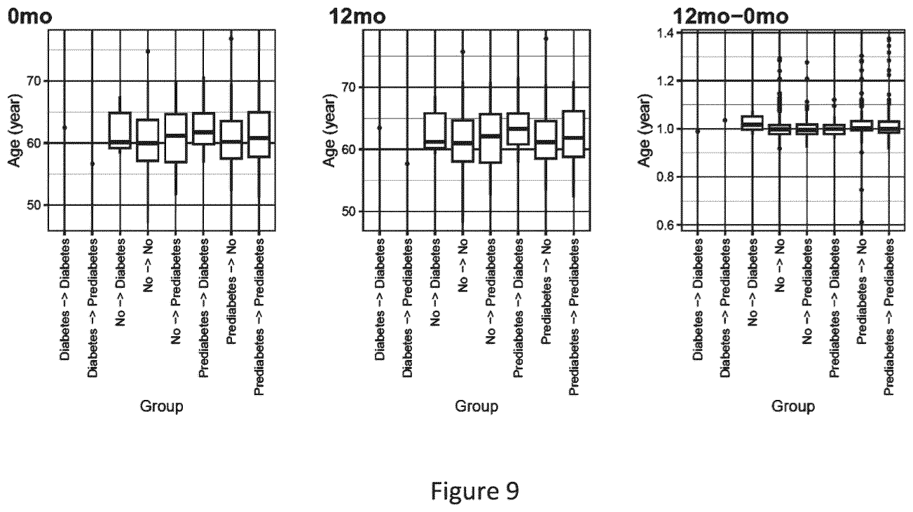

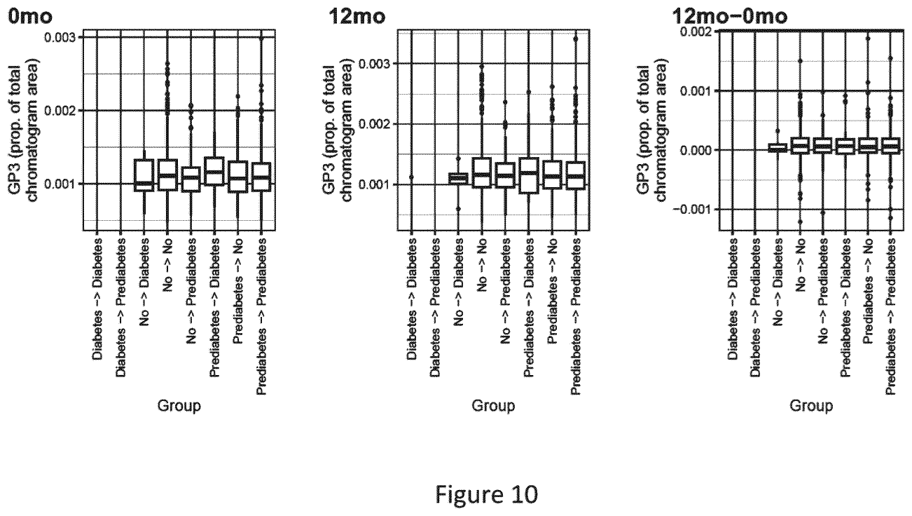

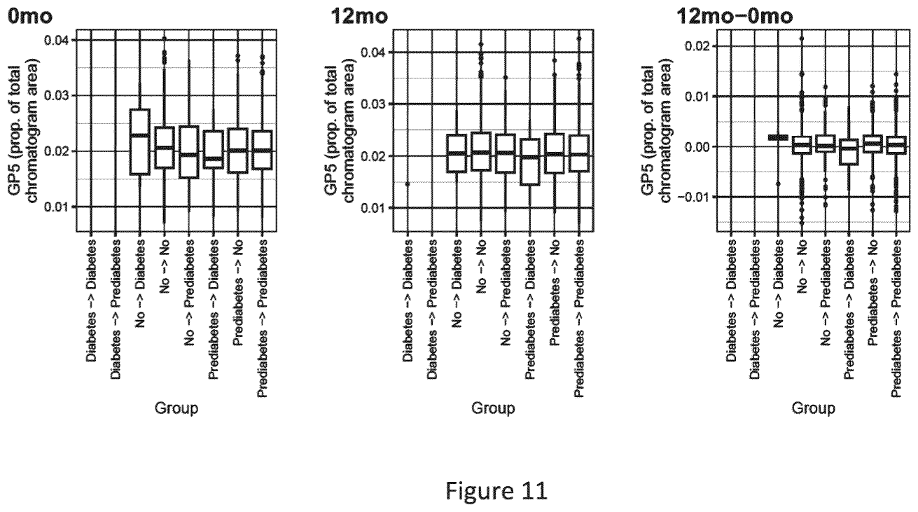

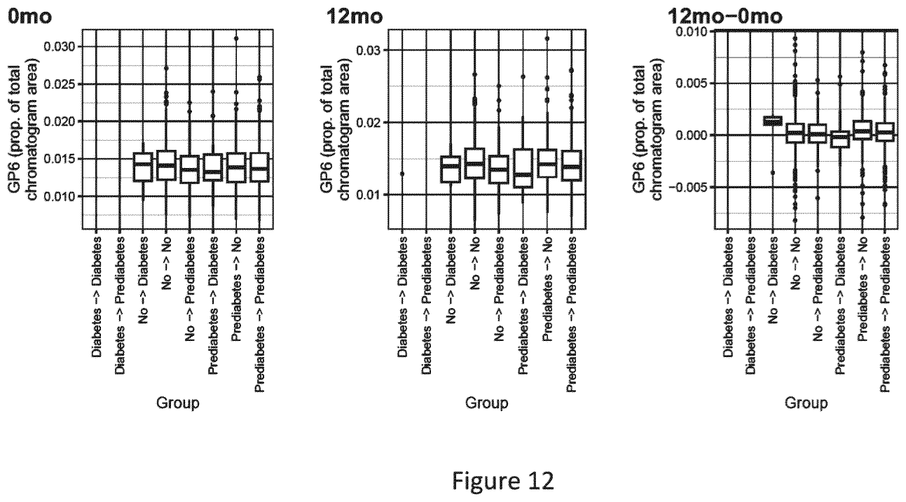

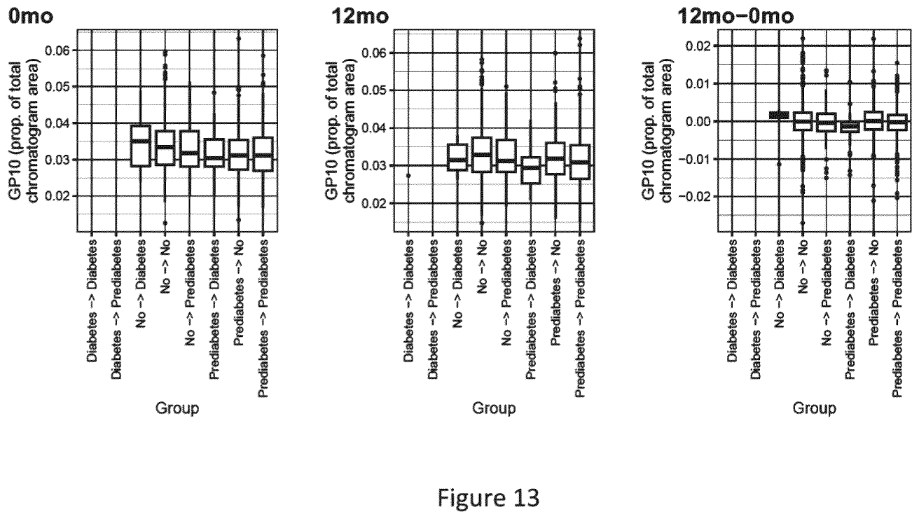

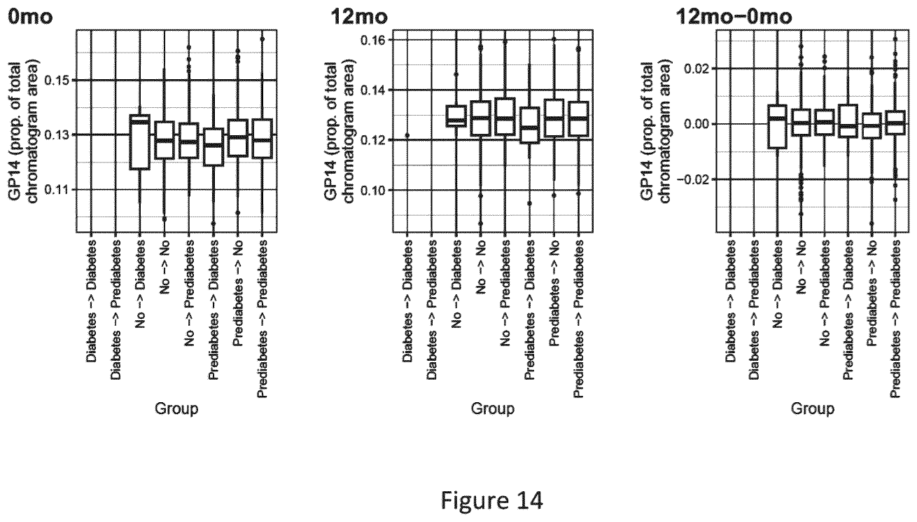

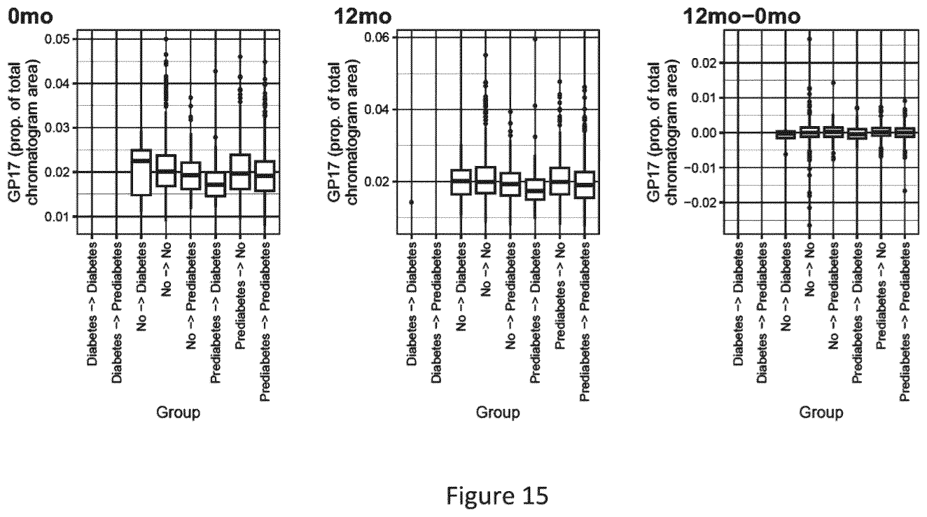

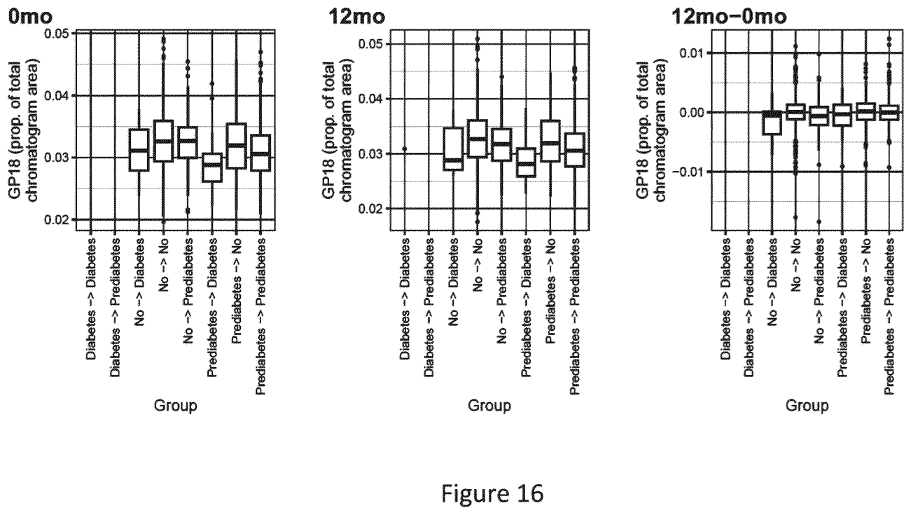

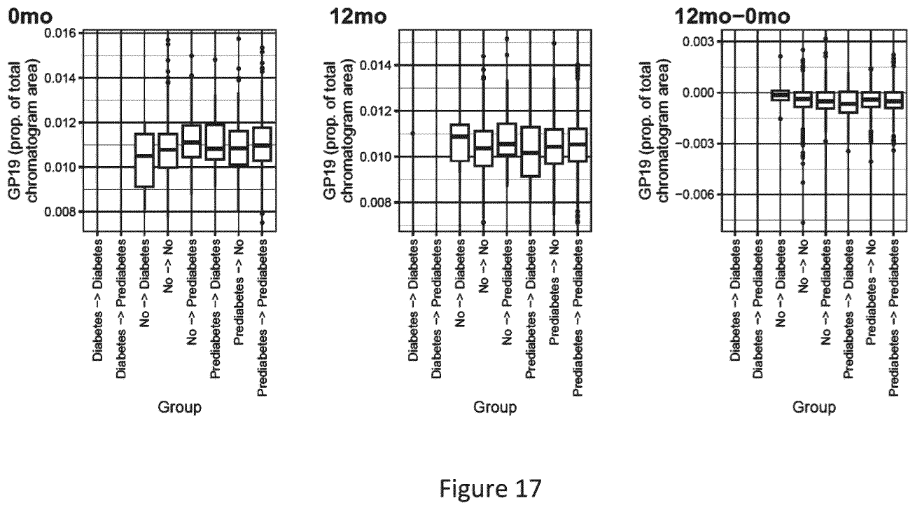

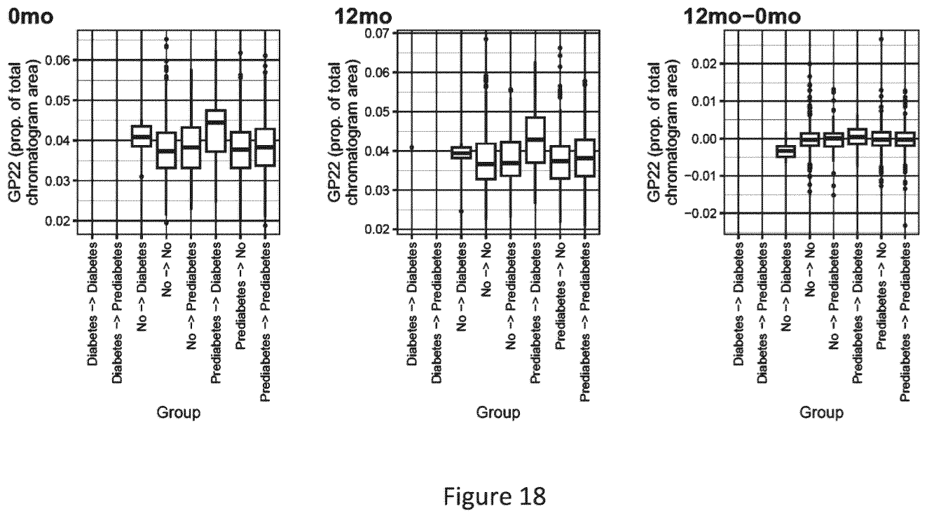

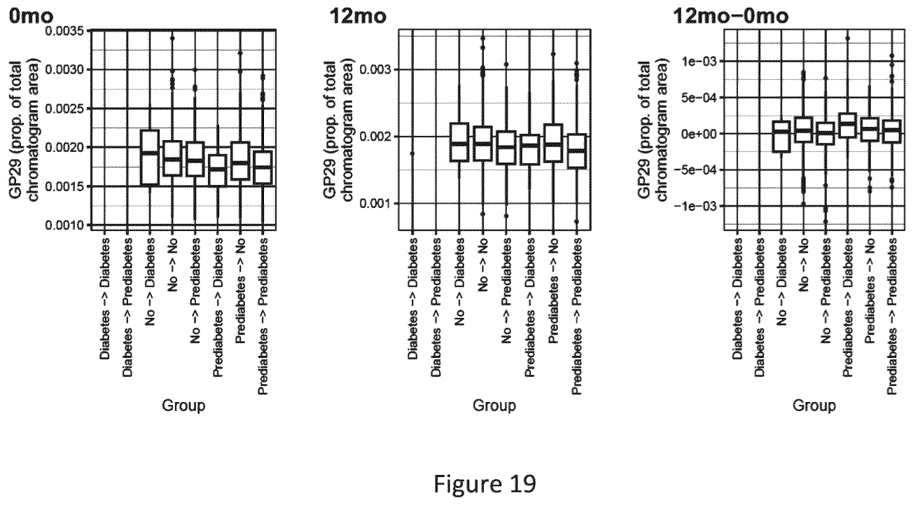

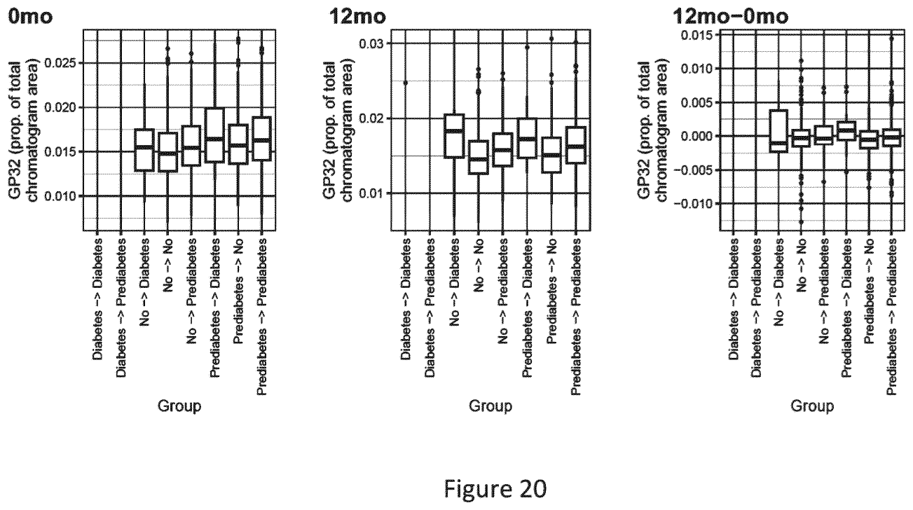

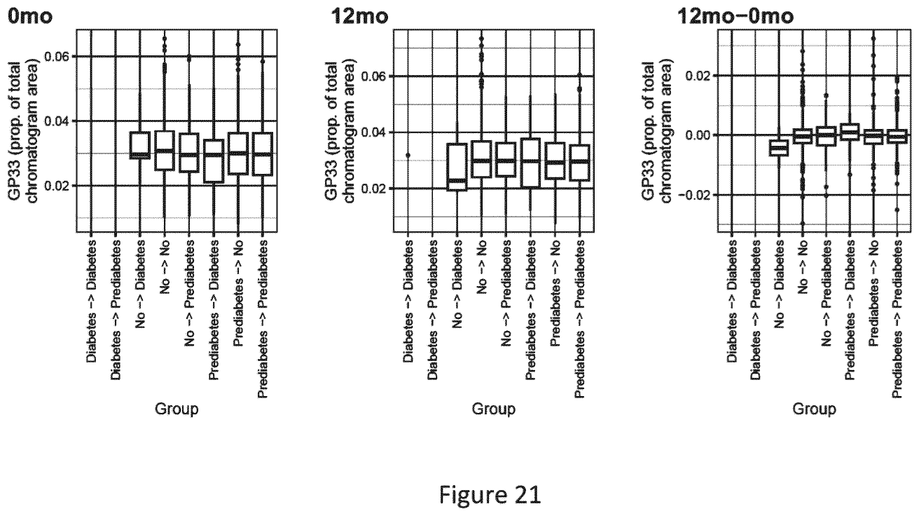

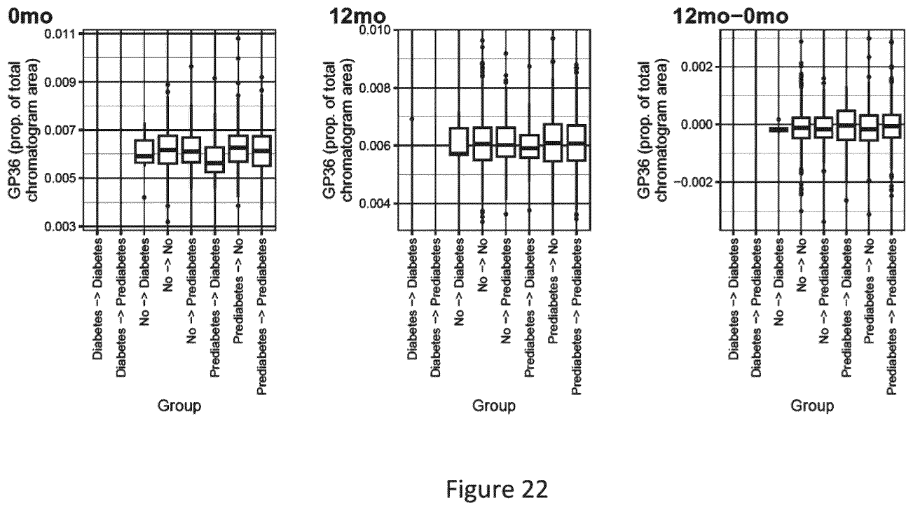

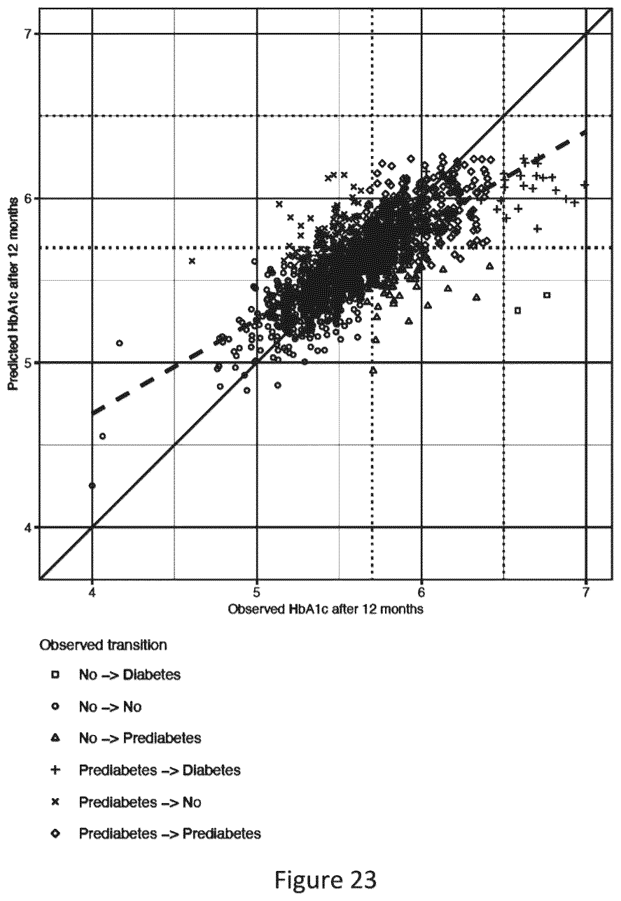

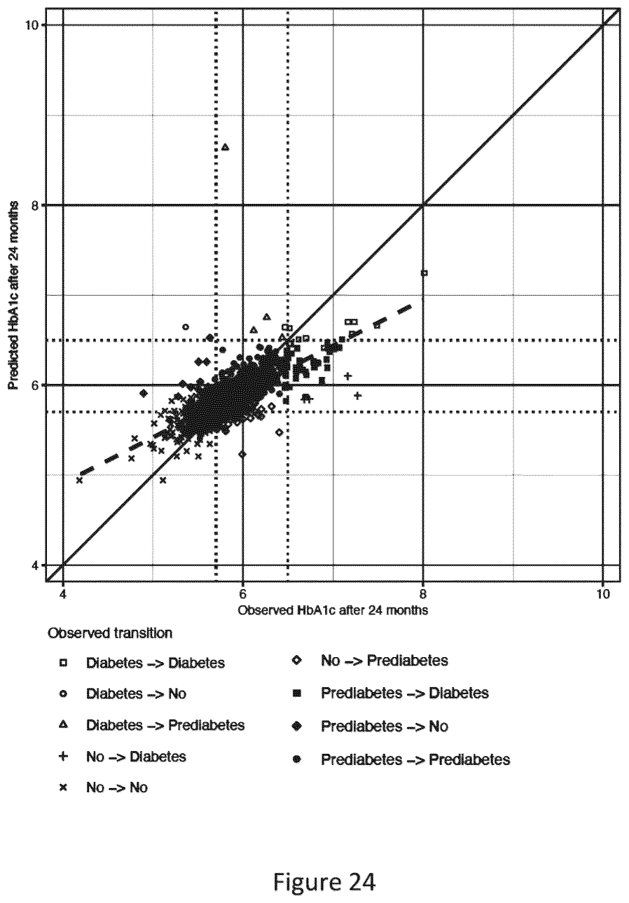

In a further embodiment of this disclosure, the determination of the total plasma protein N-glycans under the peaks GP3, GP5, GP6, GP10, GP14, GP17, GP18, GP19, GP22, GP29, GP32, GP33, and GP36 from the chromatogram in FIG. 1 can be performed by alternative quantitative analysis techniques selected from the group consisting of: MALDI-TOF mass spectrometry, liquid chromatography coupled with mass spectrometry (LC-MS), or capillary electrophoresis (CE), or by LC-MS analysis of the corresponding glycopeptides.

### Use of the Method According to the Present Disclosure

The diagnostic process according to the present invention is used for predicting the future value of HbA1c parameter in human subjects that is very likely to occur within 12 or 24 months.

A typical procedure for routine determination of future HbA1c parameter value according to the method disclosed in the present invention is described in Example 3.

### Experimental Part

### General Information

The term “room temperature” refers to a temperature interval of 20-23° C. The rotation speed of a centrifuge is expressed as the number of revolutions per minute (rpm.).

Chemicals, reagents, and accessories used in this research are purchased from the following suppliers: Igepal CA-630: Sigma-Aldrich (US); sodium dodecylsulfate (SDS): Sigma-Aldrich (US); PBS buffer: Sigma-Aldrich (US); PNGase F (10 U/μL): Promega Corporation (US); 2-aminobenzamide (2AB): Sigma-Aldrich (US); 2-picoline borane (2PB): Sigma-Aldrich (US); dimethyl sulfoxide (DMSO): Sigma-Aldrich (US); acetic acid (CH₃COOH): Merck (DE); GHP Acroprep 0.20 μm filter plate: Pall Corp. (US); acetonitrile, HPLC grade: Scharlab(ES); ethanol: Carlo Erba (IT); ultrapure water: Millipore (US); ammonium formate (HCOONH₄): Acros Organics (BE); Acquity UPLC Glycan BEH amide column, 130 Å, 1.7 μm, 2.1 mm×100 mm: Waters (US); Acquity UPLC H-Class system: Waters (US); reaction tubes ARC: Abbott Diagnostics (US); centrifuge, model 5840: Eppendorf (DE); Fume cupboard DIGIM 15 AFM: Schneider (FR); Water purification system Direct-Q 3UV: Millipore (US); analytical balance Explorer®: Ohaus Corporation (US); pH-meter FiveEasy™: Mettler Toledo (CH); precise balance JL1502-G: Mettler Toledo (CH); laboratory incubator: M. R. C.; centrifuge miniSpin: Eppendorf (DE); magnetic stirrer MR 3000 D: Heildoph (DE); Pipet-Lite XLS manual micropipette Rainin: Mettler Toledo (CH); circular shaker, model 3023: GFL; Refrigerated Vapor Traps RVT400 and vacuum pump OFP400: Thermo Scientific (US); vacuum manifold and vacuum pump: Pall (US); laboratory shaker Vortex-Genie 2: Scientific Industries (US).

### Example 1. Determination of Total Plasma Protein N-Glycans GP1-GP39 in Blood Samples

Isolation of Plasma from One or More Blood Samples that have been Collected from the Human Subject Under Examination

The isolations of blood plasma samples from human subjects were performed by the methodology known in the prior art, see literature reference 4.

### Denaturation and Release of Total Plasma Protein N-Glycans

### Denaturation

Previously prepared solutions of 4% Igepal CA-630, 2% sodium dodecyl sulfate (SDS), and 5× concentrated PBS buffer were brought to room temperature, approximately 30 minutes before starting the work.
20 μL of 20% SDS was added to the samples, and the samples were incubated for 10 minutes at 65° C.
After colling for 30 minutes at room temperature, 10 μL of Igepal was added to the samples to deactivate the excess SDS and prevent denaturation of the enzyme used for deglycosylation.
The samples were incubated for 15 minutes at room temperature.
   Deglycosylation (Release) of Glycans from Plasma Proteins.
Deglycosylation of total plasma proteins was performed using the enzyme PNGase F.
An enzyme solution was prepared, containing 10 μL of 5× concentrated PBS buffer and 0.12 μL of PNGase F (10 U/μL) per sample.
9.8 μL of the enzyme solution was added to the samples, and they were incubated for 18 hours at 37° C.
   Fluorescent Labelling with 2-Aminobenzamide (2AB) and Purification of 2AB-Derivatised Total Plasma Protein N-Glycans

### Fluorescent Labeling

Released glycans from total plasma proteins were labeled with the fluorescent dye 2-aminobenzamide (2AB).
The labeling mixture for the samples was freshly prepared. For each sample, 0.48 mg of 2AB and 1.12 mg of the reducing agent 2-picoline borane (2PB) were added in 25 μL of a dimethyl sulfoxide (DMSO)/acetic acid mixture (70:30, V/V).
25 μL of the labeling mixture was added to each sample, and the sample plate was covered with a transparent adhesive foil and incubated at room temperature for 10 minutes.
The glycan labeling reaction was carried out by incubating for 2 hours at 65° C.
After the incubation, the samples were left at room temperature for 30 minutes before proceeding with the purification of labeled glycans.

### Purification of Fluorescently Labeled Glycans

For the purification of fluorescently labeled plasma glycans from excess fluorescent dye, reagents, and proteins, a hydrophilic 0.2 μm AcroPrep wwPTFE filter plate was used with a vacuum filtration device. 100% acetonitrile, freshly prepared 70% ethanol, and 96% acetonitrile are chilled at +4° C.

### Preconditioning of WwPTFE Filter Plate

the AcroPrep wwPTFE filter plate was washed with 200 μL of 70% ethanol, 200 μL of ultrapure water (18 MΩ cm), and 200 μL of 96% acetonitrile using a vacuum device (maximum 2 inHg).

### Sample Application and Washing

After cooling the samples at room temperature for 30 minutes, 700 μL of cold 100% acetonitrile was added to each sample.
Diluted samples were transferred to the washed wwPTFE filter plate. The samples were incubated for 2 minutes, followed by vacuum drainage.
3×200 μL of cold 96% acetonitrile was added to the wwPTFE filter plate under a vacuum of a maximum of 2 inHg.
The wwPTFE filter plate was moved to a collection plate, and the last 200 μL of 96% acetonitrile was added.
Centrifugation was performed for 5 minutes at 1,000 rpm.

### Elution of Glycans

After centrifugation, the wwPTFE filter plate was moved to a 1 mL collection plate, and then 90 μL of ultrapure water was added.
Samples were incubated with mixing at room temperature for 15 minutes and then centrifuged for 5 minutes at 1, 000 rpm to elute the first fraction of glycans.
After centrifugation, another 90 μL of ultrapure water was added, incubated for 15 minutes, and centrifuged as described earlier.
180 μL of glycans were stored at-20° C. until analysis on the UPLC device.

### UPLC Analysis of Purified Fluorescently Labeled Glycans

Fluorescently labeled and purified N-glycans of total plasma proteins were analyzed using a Hydrophilic Interaction Liquid Chromatography Ultra-Performance Liquid Chromatography (HILIC-UPLC) method in a linear gradient, see Table 1. Plasma glycan standards labeled with 2AB were used as a control for the chromatographic method.

All glycan samples and standards, except ultrapure water, were prepared by mixing with acetonitrile (φ=100%) in the ratio sample:acetonitrile=30:70, V/V, by the way that prepared sample volume is for 10 μL more than injected volume (Vᵢₙⱼ). Each sample was analyzed in the following manner:
(1) ultrapure water (18 MΩ cm at 25° C.), Vᵢₙⱼ=10 μL;
(2) dextran (external standard), Vᵢₙⱼ=5 μL;
(3) internal standard of labeled and purified plasma glycans, Vᵢₙⱼ=20 μL;
(4) dextran (external standard), Vᵢₙⱼ=5 μL;
(5) prepared sample from the examined subject, Vᵢₙⱼ=20 μL;
(6) internal standard of labeled and purified plasma glycans, Vᵢₙⱼ=20 μL; and
(7) ultrapure water (18 MΩ cm at 25° C.), Vᵢₙⱼ=10 μL.

### Conditions of the Analysis

Instruments: Waters Acquity UPLC H-class consisting of a solvent manager module, a sample manager module, and an FLR fluorescence detector.
Column: Waters Acquity BEH Glycan 150×2.1 mm, 1.7 μm, 130 Å.
Mobile phase: A=100 mM ammonium formate solution (pH=4.4); B=acetonitrile; seal wash solvent=20% acetonitrile.
Excitation wavelength: 250 nm, emission wavelength: 428 nm.
Flow rate: 0.561 mL/min.
Initial gradient conditions: 30% A and 70% B.
Column temperature: 25° C.; sample temperature: 10° C.
Run time: 32.5 min for the separation of targeted peaks GP1 . . . GP39, while the rest of the time up to 55 min is employed for washing of the chromatographic column and UPLC machine.

The conditions of the gradient of solvent A and solvent B of the mobile phase and its flow are given in Table 1.

TABLE 1
The conditions of the gradient of solvent A and solvent
B of the mobile phase and its flow during UPLC analysis
of labeled and purified total plasma protein N-glycans
according to the method of the present disclosure.

| | Time | Flow | Solvent A | Solvent B | |
|---|---|---|---|---|---|
| No. | [min] | [mL/min] | [%, V/V] | [%, V/V] | Curve |
| 1 | Initial | 0.561 | 30.0 | 70.0 | Initial |
| 2 | 1.47 | 0.561 | 30.0 | 70.0 | 6 |
| 3 | 24.81 | 0.561 | 47.0 | 53.0 | 6 |
| 4 | 25.50 | 0.250 | 100.0 | 0.0 | 6 |
| 5 | 28.00 | 0.250 | 100.0 | 0.0 | 6 |
| 6 | 29.00 | 0.250 | 30.0 | 70.0 | 1 |
| 7 | 32.50 | 0.561 | 30.0 | 70.0 | 6 |
| 8 | 45.00 | 0.400 | 0.0 | 100.0 | 11 |
| 9 | 55.00 | 0.000 | 0.0 | 100.0 | 11 |

Separated glycans were detected by FLR detector at a wavelength for 2AB: λₑₓ=250 nm, λₑₘ=428 nm).

The typical chromatogram obtained by this method is presented in FIG. 1, while the retention times (RT) of thus separated total plasma protein glycan peaks GP1-GP39 are given in Table 2.

TABLE 2
Retention times (RT) for respective glycan peaks GP1-GP39 as
obtained by the UPLC analytical method described in Example
1. The corresponding UPLC chromatogram is shown in FIG. 1.

| | Glycan | RT^{a} |
|---|---|---|
| No. | peak | [min] |
| 1 | GP1 | 5.65-6.45 |
| 2 | GP2 | 6.45-7.22 |
| 3 | GP3 | 7.22-7.43 |
| 4 | GP4 | 7.43-7.87 |
| 5 | GP5 | 7.87-8.08 |
| 6 | GP6 | 8.08-8.30 |
| 7 | GP7 | 8.30-8.62 |
| 8 | GP8 | 8.62-9.00 |
| 9 | GP9 | 9.00-9.30 |
| 10 | GP10 | 9.30-9.83 |
| 11 | GP11 | 9.83-10.15 |
| 12 | GP12 | 10.15-10.62 |
| 13 | GP13 | 10.62-11.15 |
| 14 | GP14 | 11.15-11.77 |
| 15 | GP15 | 11.77-12.00 |
| 16 | GP16 | 12.00-12.55 |
| 17 | GP17 | 12.55-12.93 |
| 18 | GP18 | 12.93-13.37 |
| 19 | GP19 | 13.37-13.70 |
| 20 | GP20 | 13.70-14.25 |
| 21 | GP21 | 14.25-14.48 |
| 22 | GP22 | 14.48-14.92 |
| 23 | GP23 | 14.92-15.18 |
| 24 | GP24 | 15.18-15.52 |
| 25 | GP25 | 15.52-15.75 |
| 26 | GP26 | 15.75-16.37 |
| 27 | GP27 | 16.37-16.67 |
| 28 | GP28 | 16.67-16.93 |
| 29 | GP29 | 16.93-17.17 |
| 30 | GP30 | 17.17-17.90 |
| 31 | GP31 | 17.90-18.12 |
| 32 | GP32 | 18.12-18.37 |
| 33 | GP33 | 18.37-18.72 |
| 34 | GP34 | 18.72-18.92 |
| 35 | GP35 | 18.92-19.22 |
| 36 | GP36 | 19.22-19.65 |
| 37 | GP37 | 19.65-20.00 |
| 38 | GP38 | 20.00-20.82 |
| 39 | GP39 | 20.82-22.27 |
| — | — | — |

^{a}RT = retention time of the respective glycan peak.

The chromatograms processing that was employed during the study described in Example 2 is disclosed below:

Each chromatogram was integrated, Each i.e., separated into 39 chromatographic peaks GP1 . . . GP39 as shown in FIG. 1, either manually (for internal standards to ensure continuous verification) or by an automatic integration method (after analyzing the first part of the sample). The area percentage of each peak, representing the relative abundance of individual glycan structures, was expressed as a percentage of the total area of all peaks corresponding to glycan structures.

The glycan structures corresponding to each of the chromatographic peaks were determined by analyzing fluorescently labeled glycans using mass spectrometry.

In order to monitor the consistency of the glycan release, labeling, and purification method, aliquots of the plasma internal standard were used and analyzed together with the samples. Each plate contained five to seven standard samples. Due to the large number of samples (that were processed during the study described in Example 2) and the long duration of the analysis, the standard samples were used to monitor the influence of the analyst, temperature, solvents (mobile phase), and other conditions that may change during the analysis (referred to as batch-effect).

### Automated Integration of Profiles

High-quality preprocessing raw data is a key prerequisite for the statistical analysis of N-glycome data. The data preprocessing protocol can be divided into three parts: integration of chromatographic profiles, raw data normalization, and correction of batch effects. Profiles obtained from UPLC analysis are integrated using an algorithm for automated integration. The main characteristic of the automated integration approach is the use of a small number of manually integrated chromatographic profiles to train the model. Based on this training, the algorithm is capable of learning to mimic peak detection and integration by extracting essential features from manually preprocessed profiles and using them in an iterative comparison with the remaining profiles. The plasma N-glycan profile is integrated into said 39 peaks, GP1 . . . GP39, see FIG. 1.

### Normalization of Raw Data

In the second step of the data preprocessing process, we normalized the peak areas obtained from automated integration using the total area normalization method. The normalization procedure was necessary because signal intensities obtained from the UPLC method can vary up to 100-fold due to experimental reasons.

### Batch Effects Correction

In the third step of the data preprocessing process, we applied batch effects correction to the previously normalized measurements. The batch effects correction procedure is essential because laboratory conditions during the experiment can vary significantly. Before the correction, the normalized measurements were log-transformed due to the multiplicative nature of the batch effects themselves. The batch effects correction was carried out using an empirical Bayes model.

### Example 2. The Study of Total Plasma Protein N-Glycans in a Large Population of Subjects in Terms of Determining Future Events Regarding the Regulation of Glycemia: Prediabetes or Type 2 Diabetes (T2D)

The aim of this study was to investigate the relationship between total plasma protein N-glycans and current HbA1c value, and the incidence of future development of prediabetes or diabetes. The study was performed on a large population of human subjects (N=1826 participants) that have diabetes indication in their family and were monitored at starting time point and after 12 months regarding their glycemic status against total plasma protein N-glycome and HbA1c value. The participants were categorized based on their diagnosis at time point 0 (measurement 1) and time point 1 (measurement after 12 months), and divided into the following groups according to the glycemic status expressed through HbA1c value:
(i) those that had diabetes at the 0-month time point and remained in the diabetes condition after 12 months (a sub-group marked with: □; Diabetes->Diabetes; 1 participant),
(ii) those that had diabetes at the 0-month time point but have become healthy after 12 months (a sub-group marked with: Diabetes->No; 0 participants),
(iii) those that had diabetes at the 0-month time point but have developed prediabetes after 12 months (a sub-group marked with: Δ; Diabetes->Prediabetes; 0 participants),
(iv) those that were healthy at the 0-month time point but have developed diabetes after 12 months (a sub-group marked with: +; No->Diabetes; 6 participants),
(v) those that were healthy at the 0-month time point and remained healthy after 12 months (a sub-group marked with: x; No->No; 801 participants),
(vi) those that were healthy at the 0-month time point but have developed prediabetes after 12 months ((a sub-group marked with: ⋄; No->Prediabetes; 113 participants),
(vii) those that had prediabetes at the 0-month time point but have developed diabetes after 12 months (a sub-group marked with: ▪; Prediabetes->Diabetes; 30 participants),
(viii) those that had prediabetes at the 0-month time point and have become healthy after 12 months (a sub-group marked with: ♦; Prediabetes->No; 282 participants), and
(ix) those that had prediabetes at the 0-month time point and remained in the prediabetes condition after 12 months (a sub-group marked with: •; Prediabetes->Prediabetes; 593 participants).

The categories were assigned based on the HbA1c values at the time of measurement at point 0, and the groups were labeled with the corresponding terms as follows:
(1) with HbA1c value<5.7, labeled as: No (healthy);
(2) with HbA1c value from 5.7-6.5, labeled as: Prediabetes; and
(3) with HbA1c value>6.5, labeled as: Diabetes.

During the inclusion of participants in the study, the inclusion and exclusion criteria were defined. All participants included in the study were between 55 and 69 years old and had at least one first-degree family member (mother, father, sister, brother, or children) with diabetes, but the participants themselves did not have diabetes. Additionally, participants did not have the following conditions: autoimmune diseases (multiple sclerosis, Crohn's disease, psoriasis, rheumatoid arthritis, Hashimoto's disease, systemic lupus erythematosus, etc.), hematological diseases of red blood cells (hemolytic anemia, thalassemia, etc.), malignant diseases (except for basal cell carcinoma), they were not taking corticosteroids or antipsychotic medications. These exclusion criteria were implemented because they can affect glycan changes, while the inclusion criteria were introduced to obtain a statistically significant number of participants with diabetes over a period of 3 years.

### Summarized Results

Biochemical values related to glycemia and recorded anthropometric characteristics of blood glucose concentration [mmol/L] were measured for the above-mentioned groups of participants. The processed results are presented graphically in FIGS. 2-9: variation of blood glucose concentration [mmol/L], see FIG. 2; variation of HbA1c [%], see FIG. 3; variation of HbA1c [mmol/mol], see FIG. 4; variation of body weight [kg], see FIG. 5; variation of body height [cm], see FIG. 6; variation of waist [cm], see FIG. 7; variation of body mass index (BMI) [kg/m²], see FIG. 8; and for variation of age [years], see FIG. 9.

Similarly, the median, minimum, maximum and quartiles Q1 and Q3 of the respective total plasma protein N-glycan levels GP1 . . . GP39 were determined for all the above-mentioned groups of participants.

After associating corresponding glycan values to each group of participants based on their initial (time point 0) and final diagnosis (after 12 months), a comparison of glycan values among these groups was conducted, and certain glycans that showed statistically significant differences among the mentioned groups were identified.

In other words, glycan values measured at time point 0 were compared between groups based on their diagnosis status after 12 months (predictive value). The obtained results indicate that there are glycans that statistically differ at point 0 among the examined groups of participants, considering their diagnosis (according to HbA1c value) they will have after 12 months.

Glycans that reached statistical significance (p<0.005 after correction) are used for the generation of the numerical model for the prediction of the future value of HbA1c according to the present disclosure. The appearance of these glycans in groups of participants at:
a) point 0 with respect to the diagnosis that will occur after 12 months,
b) at the point after 12 months when the diagnosis actually happens, and
c) the magnitude of their difference between measurements after 12 months and at point 0 is shown in FIGS. 9-22.

The processed results for only relevant glycan peaks that show a statistically significant correlation, namely, glycan peaks: GP3, GP5, GP6, GP10, GP14, GP17, GP18, GP19, GP22, GP29, GP32, GP33, and GP36 in groups of participants involved in the study are presented graphically in FIGS. 10-22:
variation of GP3, see FIG. 10;
variation of GP5, see FIG. 11;
variation of GP6, see FIG. 12;
variation of GP10 see FIG. 13;
variation of GP14, see FIG. 14;
variation of GP17, see FIG. 15;
variation of GP18, see FIG. 16;
variation of GP19, see FIG. 17;
variation of GP22, see FIG. 18;
variation of GP29, FIG. 19;
variation of GP32, see FIG. 20;
variation of GP33 see FIG. 21; and for variation of GP36, see FIG. 22.

From thus obtained results, two prediction models for glycemic status expressed as predicted HbA1c parameter value after 12 months [HbA1c(12 months)] and after 24 months [HbA1c(24 months] have been developed.

The first one, for prediction of HbA1c(12 months) is based on the seven key glycans GP3, GP14, GP17, GP18, GP19, GP29, and GP36 as well as current HbA1c (cHbA1c) value, measured at point 0. The model works by predicting the HbA1c value that the examined person will have after 12 months and then assigns the subject to one of the following groups: (1) Healthy, (2) Prediabetes, or (3) Diabetes, which is the predicted diagnosis for the person after 12 months. The model demonstrated a diagnostic prediction success rate of 78.3% in the investigated population. Additionally, the model shows the probability of developing the predicted diagnosis in comparison to the other two diagnoses (based on the possible range of predicted HbA1c values) as a percentage (8).

For example, individual X had their HbA1c value measured at point 0, along with all glycans, which were then included in the model (formula below). The model predicted that after 12 months, individual X's HbA1c would be 5.8, categorizing him into the prediabetic group with a 70% probability for that specific diagnosis, and a 30% probability for a healthy diagnosis, while the probability of developing diabetes in the next 12 months for the examined subject is almost negligible.

The model's overall performance for the entire population is shown in FIG. 23.

The said HbA1c(12 months) prediction model, and the probability of belonging to a specific group based on the formula for a normal distribution, according to the present disclosure is shown below:

$$HbA 1 c ( 12 months ) = 0.6695 + 0.7857 \cdot HbA 1 c ( 0 ) + 0.0362 \cdot logit ( GP 3 ) ++ ( - 0.0897 \cdot logit ( GP 14 ) ) + ( - 0.0614 \cdot logit ( GP 17 ) ) ++ ( - 0.0596 \cdot logit ( GP 18 ) ) + ( 0.0934 \cdot logit ( GP 19 ) ) ++ ( - 0.0339 \cdot logit ( GP 29 ) ) + ( - 0.0651 \cdot logit ( GP 36 ) )$$

where:
HbA1c(12 months) is the predicted HbA1c value for the examined subject for 12 months in the future,
HbA1c(0) is the current HbA1c value<cHbA1c> of the examined subject at the time of examination,
and
the calculated value for HbA1c(12 months) reveals the following conclusion for the examined subject:
A. HbA1c(12 months) is <5.7, then the examined subject will be healthy,
B. HbA1c(12 months) is 5.7-6.7, then the examined subject will develop prediabetes, with the prediction probability at the level of 78%, and 22% probability that will be healthy, and
C. HbA1c(12 months) is >6.7, then the examined subject will develop diabetes, with the prediction probability at the level of 78%, and 22% probability that will develop prediabetes.

Additionally, the second model for the prediction of glycemic status after 24 months, expressed as the HbA1c(24 months) parameter, was developed based on initial measurements and measurements after 12 months.

The model works by predicting the HbA1c value that an examined subject will have after 24 months by monitoring the person's progress during the first 12 months and then assigning the individual to one of the following groups: (1) Healthy, (2) Prediabetes, or (3) Diabetes, which is the predicted diagnosis for the person after 24 months. The model demonstrated a diagnostic prediction success rate of a 70% in the investigated population. Additionally, the model shows the probability of developing the predicted diagnosis in comparison to the other two diagnoses (based on the possible range of predicted HbA1c values) as a percentage.

This HbA1c(24 months) prediction model, and the probability of belonging to a specific group based on the formula for a normal distribution, according to the present disclosure is shown below:

$$HbA 1 c ( 24 months ) = 1.0314 \cdot Intercept + 0.6569 \cdot HbA 1 c ( prop . before ) ++ ( - 0.1167 \cdot logit ( GP 10 ) ) + ( - 0.0717 \cdot logit ( GP 18 ) ) ++ ( 0.1109 \cdot logit ( GP 32 ) ) + ( - 0.0578 \cdot logit ( GP 33 ) ) ++ ( - 0.1456 \cdot logit ( GP 36 ) ) + ( 0.2648 \cdot logit ( GP5\_ 12 -- GP5\_ 0 ) ) + ( - 0.4114 \cdot logit ( GP6\_ 12 - GP6\_ 0 ) ) ++ ( 0.0963 \cdot logit ( GP22\_ 12 - GP22\_ 0 ) ) ++ ( - 0.0885 \cdot logit ( GP33\_ 12 - GP33\_ 0 )$$

where:
Intercept is a value on the y-axis when all factors in the said model are equal to zero,
HbA1c(prop.before) is a proportion of glycated HbA1c expressed in percentage <%> to total HbA1c,
GPX_Y is a value of the result of the corresponding glycan peak GP of number X, GPX, determined in the respective time point expressed in months, Y, which can be: Y=0 or Y=12 months,
   and
the calculated value for HbA1c(24 months) reveals the following conclusion for the examined subject:
A. HbA1c(24 months) is <5.7, then the examined subject will be healthy,
B. HbA1c(24 months) is 5.7-6.7, then the examined subject will develop prediabetes, with the prediction probability at the level of 70%, and 30% probability that will be healthy, and
C. HbA1c(24 months) is >6.7, then the examined subject will develop diabetes, with the prediction probability at the level of 70%, and 30% probability that will develop prediabetes.

The model's overall performance for the entire population is shown in FIG. 24.

### Statistics

The statistical methodology applied in performing the study and during the development of the numerical models according to the present disclosure is as follows; for each sample, raw UPLC data were normalized by dividing the peak area by the total chromatogram area. Normalized areas of glycan peaks were then logit transformed and batch corrected using the ComBat method (R package sva), see literature reference 21:
21) J. T. Leek, W. E. Johnson, H. S. Parker, A. E. Jaffe, J. D. Storey: The SVA package for removing batch effects and other unwanted variation in high-throughput experiments, Bioinformatics 28 (2012) 882-883.

After batch correction, areas were back-transformed and glycan contents were calculated. Subjects were classified into 6 categories based on HbA1c values at two-time points, see Table 3.

TABLE 3
Definition of categories with HbA1c levels in two-time points.

| | Category | HbA1c value at | HbA1c value at |
|---|---|---|---|
| No. | (T2D status) | baseline | 12 months follow up |
| 1 | No -> No | <0, 5.7> | <0, 5.7> |
| 2 | No -> Prediabetes | <0, 5.7> | [5.7, 6.5> |
| 3 | No -> Diabetes | <0, 5.7> | [6.5, ∞> |
| 4 | Prediabetes -> No | [5.7, 6.5> | <0, 5.7> |
| 5 | Prediabetes -> Prediabetes | [5.7, 6.5> | [5.7, 6.5> |
| 6 | Prediabetes -> Diabetes | [5.7, 6.5> | [6.5, ∞> |

T2D = Type 2 diabetes

To estimate the predictive value of glycome measured in the baseline, differences in categories were analyzed using general linear modeling. Logit-transformed glycans were dependent variables, while categories of age and sex were modeled as independent variables.

Models for prediction of the 12- and 24-months follow-up HbA1c values were designed using general linear modeling coupled with lasso penalization for variable selection, see literature reference 22, and cross-validation error estimation to avoid overfitting, see literature reference 23:
22) J. Friedman, T. Hastie, R. Tibshirani: Regularization Paths for Generalized Linear Models via Coordinate Descent, J. Stat. Softw. 33 (2010) 1-22.
23) M. Kuhn: Building Predictive Models in R Using the Caret Package, J. Stat. Softw. 28 (2008) 1-26.

In the first model 12-month follow-up HbA1c was defined as the dependent variable while all directly measured glycans peaks, as well as HbA1c measured at baseline, were set as independent variables. In the second model, 24-month follow-up HbA1c was defined as the dependent variable while all directly measured glycans peaks, 12-month to baseline differences in glycan peaks, and HbA1c measured at 12-month follow-up were set as independent variables. Prior to modeling all glycan peaks were logit transformed to normalize their distributions. The false discovery rate was controlled by adjusting p values by the Benjamini-Hochberg method modified by Li and Ji, see literature reference 24:
24) J. Li, L. Ji: Adjusting multiple testing in multilocus analyses using the eigenvalues of a correlation matrix. Heredity (Edinb.) 95 (2005) 221-227.

### Example 3. A Typical Procedure for Routine Determination of Future HbA1c Parameter Value According to the Method Disclosed in the Present Invention

The typical procedure for performing the future HbA1c value assessment method of the subject being examined, according to the present disclosure, is as follows:
a) isolation of plasma from one or more blood samples that have been collected from the human subject under examination; the isolation procedure is a common clinical practice known in the art, e.g., see literature reference 4;
b) denaturation and release of total plasma protein N-glycans from total plasma proteins by deglycosylation;
c) fluorescent derivatization with 2-aminobenzamide (2AB) and a reducing agent for reductive amination, optionally using a complex of picoline borane (BH₃·NC₅H₄-2-CH₃) or sodium cyanoborohydride (NaBH₃CN);
d) quantitative analysis of thus derivatized glycans by ultra-performance liquid chromatography (UPLC), wherein numerical values of relative areas under respective glycan peaks GP3, GP5, GP6, GP10, GP14, GP17, GP18, GP19, GP22, GP29, GP32, GP33, and GP36 within the corresponding UPLC chromatogram are obtained as the numerical values {GP3, GP5, GP6, GP10, GP14, GP17, GP18, GP19, GP22, GP29, GP32, GP33, and GP36};
e) as well as analysis of the current HbA1c parameter value (cHbA1c) from the same blood samples that have been collected from the human subject under examination; the determination of HbA1c parameter is a common clinical practice, e.g., see literature reference 20;
f) where numerical results of relative areas under the selected glycan peaks GP3, GP5, GP6, GP10, GP14, GP17, GP18, GP19, GP22, GP29, GP32, GP33, and GP36, that correspond to values {GP3, GP5, GP6, GP10, GP14, GP17, GP18, GP19, GP22, GP29, GP32, GP33, and GP36} calculated in step d, and numerical result of the cHbA1c parameter value obtained in step e, are included in a model for calculation of the predictive value of the HbA1c parameter in the future:

$$HbA 1 c ( 12 months ) = 0.6695 + 0.7857 \cdot HbA 1 c ( 0 ) + 0.0362 \cdot logit ( GP 3 ) ++ ( - 0.0897 \cdot logit ( GP 14 ) ) + ( - 0.0614 \cdot logit ( GP 17 ) ) ++ ( - 0.0596 \cdot logit ( GP 18 ) ) + ( 0.0934 \cdot logit ( GP 19 ) ) ++ ( - 0.0339 \cdot logit ( GP 29 ) ) + ( - 0.0651 \cdot logit ( GP 36 ) )$$

where:
HbA1c(12 months) is the predicted HbA1c value for the examined subject for 12 months in the future,
HbA1c(0) is the current HbA1c value<cHbA1c> of the examined subject at the time of examination,
   and
the calculated value for HbA1c(12 months) reveals the following conclusion for the examined subject:
A. HbA1c(12 months) is <5.7, then the examined subject will be healthy,
B. HbA1c(12 months) is 5.7-6.7, then the examined subject will develop with prediabetes, the prediction probability at the level of 78%, and 22% probability that will be healthy, and
C. HbA1c(12 months) is >6.7, then the examined subject will develop diabetes, with the prediction probability at the level of 78%, and 22% probability that will develop prediabetes.

Alternatively, numerical results of relative areas under the selected glycan peaks GP3, GP5, GP6, GP10, GP14, GP17, GP18, GP19, GP22, GP29, GP32, GP33, and GP36, that correspond to values {GP3, GP5, GP6, GP10, GP14, GP17, GP18, GP19, GP22, GP29, GP32, GP33, and GP36} calculated in step d, and numerical result of the cHbA1c parameter value obtained in step e, are included in a model for calculation of the predictive value of the HbA1c parameter in the future:

$$HbA 1 c ( 24 months ) = 1.0314 \cdot Intercept + 0.6569 \cdot HbA 1 c ( prop . before ) ++ ( - 0.1167 \cdot logit ( GP 10 ) ) + ( - 0.0717 \cdot logit ( GP 18 ) ) ++ ( 0.1109 \cdot logit ( GP 32 ) ) + ( - 0.0578 \cdot logit ( GP 33 ) ) ++ ( - 0.1456 \cdot logit ( GP 36 ) ) + ( 0.2648 \cdot logit ( GP5\_ 12 -- GP5\_ 0 ) ) + ( - 0.4114 \cdot logit ( GP6\_ 12 - GP6\_ 0 ) ) ++ ( 0.0963 \cdot logit ( GP22\_ 12 - GP22\_ 0 ) ) ++ ( - 0.0885 \cdot logit ( GP33\_ 12 - GP33\_ 0 )$$

where:
Intercept is a value on the y-axis when all factors in the said model are equal to zero,
HbA1c(prop.before) is a proportion of glycated HbA1c expressed in percentage <%> to total HbA1c,
GPX_Y is a value of the result of the corresponding glycan peak GP of number X, GPX, determined in the respective time point expressed in months, Y, which can be: Y=0 or Y=12 months,
   and
the calculated value for HbA1c(24 months) reveals the following conclusion for the examined subject:
A. HbA1c(24 months) is <5.7, then the examined subject will be healthy,
B. HbA1c(24 months) is 5.7-6.7, then the examined subject will develop prediabetes, with the prediction probability at the level of 70%, and 30% probability that will be healthy, and
C. HbA1c(24 months) is >6.7, then the examined subject will develop diabetes, with the prediction probability at the level of 70%, and 30% probability that will develop prediabetes.

The detailed experimental procedures for steps b-d are described in Example 1.

### INDUSTRIAL APPLICABILITY

The present invention discloses a method for determining future HbA1c value as a parameter for assessment of future risk of diabetes development in human subjects, from one or more blood samples. In this manner, the industrial applicability of the present invention is obvious.

### Abbreviations

The nomenclature of total plasma protein N-glycans, e.g., FA1, A2, A2B, etc., is derived according to the rules of the Oxford nomenclature. The meaning of the abbreviations used is as follows:
2AB=2-aminobenzamide;
BMI=body mass index;
CI=confidence interval;
DMSO=dimethyl sulfoxide, a solvent;
GP1 . . . GP39=peaks of total plasma protein N-glycans in UPLC analytical method according to the present invention;
HbA1c=glycated hemoglobin; a diagnostic parameter which described average blood glucose levels for the last two-to-three months;
HILIC=hydrophilic interaction liquid chromatography;
2PB=2-picoline borane;
PBS phosphate-buffered saline, a buffer solution;
PNGase F=enzyme peptide-N4-(N-acetyl-beta-glucosaminyl) asparagine amidase F;
rpm=revolutions per minute;
r.t.=room temperature;
RT=retention time (tr); retention time of the respective glycan peak in e.g., UPLC chromatogram;
T2D=type 2 diabetes (mellitus);
UPLC=ultra-performance liquid chromatography.

1. A method for prediction of future glycated hemoglobin <HbA1c> parameter value in a human subject comprises performing an analysis process of N-glycans of general formulae Ia-If, bound to plasma proteins, 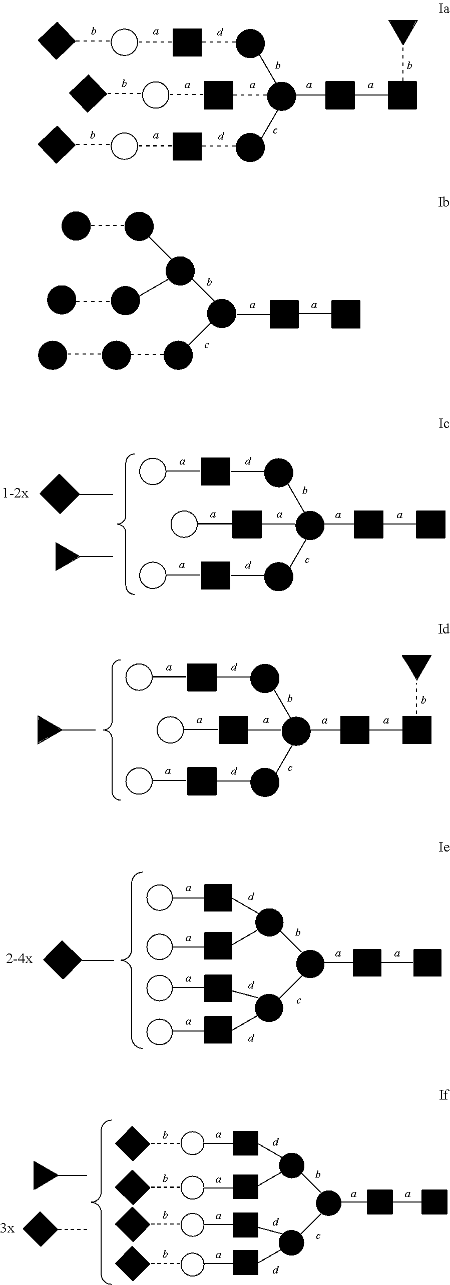
where symbols in Ia-If denote monomeric sugar units below:
▪ N-acetylglucosamine
▾ fucose
● mannose
◯ galactose
♦ sialic acid
and where letters a-d in Ia-If determine types of glycoside bonds of said N-glycans Ia-If:
a=β(1-4)
b=α(1-6)
c=α(1-3)
d=β(1-2)
where the method comprises following steps:
a) isolation of plasma from one or more blood samples that have been collected from the human subject under examination,
b) denaturation and release of the glycans from total plasma proteins by deglycosylation,
c) fluorescent derivatization of the glycans with 2-aminobenzamide <2AB> and a reducing agent for reductive amination, optionally using a complex of picoline borane <BH₃·NC₅H₄-2-CH₃> or sodium cyanoborohydride <NaBH₃CN>: 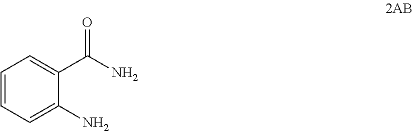
d) quantitative analysis of thus derivatized glycans by ultra-performance liquid chromatography <UPLC>, wherein numerical values of relative areas under respective glycan peaks GP3, GP5, GP6, GP10, GP14, GP17, GP18, GP19, GP22, GP29, GP32, GP33, and GP36 within the corresponding UPLC chromatogram are obtained as the numerical values {GP3, GP5, GP6, GP10, GP14, GP17, GP18, GP19, GP22, GP29, GP32, GP33, and GP36}, wherein the glycan peaks GP3, GP5, GP6, GP10, GP14, GP17, GP18 GP19, GP22, GP29, GP32, GP33, and GP36 belong to structures given in the table:

| Glycan nomenclature/ | | |
|---|---|---|
| composition | Code | Structure |
| A2[6]BG1/H4N5 | GP3 | [symbol] |
| FA2[3]G1/H4N4F1 | GP5 | [symbol] |
| FA2[ 6] BG1/H4N5F1 | GP6 | [symbol] |
| FA2G2/H5N4F1 | GP10 | [symbol] |
| A2G2S1/H5N4S1 | GP14 | [symbol] |
| FA2BG2S1/H5N5F1S1 | GP17 | [symbol] |
| A2G2S2/H5N4S2 | GP18 | [symbol] |
| FA2G2S2/H5N4F1S2 | | |
| M9/H9N2 | GP19 | [symbol] |
| FA2G2S2/H5N4F1S2 | GP22 | [symbol] |
| A3G3S3/H6N5S3 | GP29 | [symbol] |
| A3F1G3S2/H6N5F1S2 | | [symbol] |
| A3G3S3/H6N5S3 | GP32 | [symbol] |
| A4G4S2/H7N6S2 | | [symbol] |
| A3F1G3S3/H6N5F1S3 | GP33 | [symbol] |
| A4G4S3/H7N6S3 | GP36 | [symbol] |
| A4F1G4S3/H7N6F1S3 | | [symbol] |

e) analysis of a current HbA1c parameter value<cHbA1c> from the same blood samples that have been collected from the human subject under examination,
f) where numerical results of relative areas under the selected glycan peaks GP3, GP5 GP6, GP10, GP14, GP17, GP18, GP19, GP22, GP29, GP32, GP33, and GP36, that correspond to values {GP3, GP5, GP6, GP10, GP14, GP17, GP18, GP19, GP22, GP29, GP32, GP33, and GP36} calculated in step d, and numerical result of the cHbA1c parameter value obtained in step e, are included in a model for calculation of the predictive value of the HbA1c parameter in the future, which is a function of the thirteen glycan arguments and cHbA1c parameter value:
HbA1c(T)=HbA1c((GP3,GP5,GP6,GP10,GP14,GP17,GP18,GP19,GP22,GP29,GP32,GP33,GP36),cHbA1c)
where HbA1c (T) parameter means HbA1c value in time point T, having a value of 12 or 24 months in the future, and
g) determination of the predictive, future HbA1c parameter value, HbA1c(12 months) and/or HbA1c(24 months) and linking of the said parameter with probability that the examined subject will develop prediabetes, diabetes, or remain healthy after the said period of 12 or 24 months.

2. The method according to claim 1, further comprising: obtaining the future HbA1c parameter value model via statistical data analysis performed after a prospective study that determines variation of quantitative total plasma protein N-glycans {GP1, . . . , GP39} content and current HbA1c <cHbA1c> parameter value in the blood plasma in the following sub-groups of participants included in the study:
(i) those that had diabetes at 0-month time point and remained in the diabetes condition after 12 months,
(ii) those that had diabetes at the 0-month time point but have become healthy after 12 months,
(iii) those that had diabetes at the 0-month time point but have developed prediabetes after 12 months,
(iv) those that were healthy at the 0-month time point but have developed diabetes after 12 months,
(v) those that were healthy at the 0-month time point and remained healthy after 12 months,
(vi) those that were healthy at the 0-month time point but have developed prediabetes after 12 months,
(vii) those that had prediabetes at the 0-month time point but have developed diabetes after 12 months,
(viii) those that had prediabetes at the 0-month time point and have become healthy after 12 months, and
(ix) those that had prediabetes at the 0-month time point and remained in the prediabetes condition after 12 months,
wherein the set of predictive total plasma protein N-glycans are determined using regression models that are corrected for multiple confounders, including age, blood glucose concentration, HbA1c parameter value, weight, height, waist, body mass index <BMI>, from which total plasma protein N-glycome and cHbA1c parameter value-based predictive model is constructed based on the determined total plasma protein N-glycans and CHbA1c value construct.

3. The method according to claim 1, where the future HbA1c value prediction model is given with the formula:
$$HbA 1 c ( 12 months ) = 0.6695 + 0.7857 \cdot HbA 1 c ( 0 ) + 0.0362 \cdot logit ( GP 3 ) ++ ( - 0.0897 \cdot logit ( GP 14 ) ) + ( - 0.0614 \cdot logit ( GP 17 ) ) ++ ( - 0.0596 \cdot logit ( GP 18 ) ) + ( 0.0934 \cdot logit ( GP 19 ) ) ++ ( - 0.0339 \cdot logit ( GP 29 ) ) + ( - 0.0651 \cdot logit ( GP 36 ) )$$
where:
HbA1c(12 months) is the predicted HbA1c value for the examined subject for 12 months in the future,
HbA1c(0) is the current HbA1c value<cHbA1c> of the examined subject at the time of examination,
and
the calculated value for HbA1c(12 months) reveals following conclusion for the examined subject:
A. HbA1c(12 months) is <5.7, then the examined subject will be healthy,
B. HbA1c(12 months) is 5.7-6.7, then the examined subject will develop prediabetes, with the prediction probability at the level of 788, and 22% probability that will be healthy, and
C. HbA1c(12 months) is >6.7, then the examined subject will develop diabetes, with the prediction probability at the level of 78%, and 22% probability that will develop prediabetes.

4. The method according to claim 1, where the future HbA1c parameter value prediction model is given with the formula:
$$HbA 1 c ( 24 months ) = 1.0314 \cdot Intercept + 0.6569 \cdot HbA 1 c ( prop . before ) ++ ( - 0.1167 \cdot logit ( GP 10 ) ) + ( - 0.0717 \cdot logit ( GP 18 ) ) ++ ( 0.1109 \cdot logit ( GP 32 ) ) + ( - 0.0578 \cdot logit ( GP 33 ) ) ++ ( - 0.1456 \cdot logit ( GP 36 ) ) + ( 0.2648 \cdot logit ( GP5\_ 12 -- GP5\_ 0 ) ) + ( - 0.4114 \cdot logit ( GP6\_ 12 - GP6\_ 0 ) ) ++ ( 0.0963 \cdot logit ( GP22\_ 12 - GP22\_ 0 ) ) ++ ( - 0.0885 \cdot logit ( GP33\_ 12 - GP33\_ 0 )$$
where:
Intercept is a value on the y-axis when all factors in the said model are equal to zero,
Hba1c(prop.before) is a proportion of glycated HbA1c expressed in percentage <%> to total HbA1c,
GPX_Y is a value of the result of the corresponding glycan peak GP of number X, GPX, determined in the respective time point expressed in months, Y, which can be: Y=0 or Y=12 months,
and
the calculated value for HbA1c(24 months) reveals the following conclusion for the examined subject:
A. HbA1c(24 months) is <5.7, then the examined subject will be healthy,
B. HbA1c(24 months) is 5.7-6.7, then the examined subject will develop with prediabetes, the prediction probability at the level of 70%, and 30% probability that will be healthy, and
C. HbA1c(24 months) is >6.7, then the examined subject will develop diabetes, with the prediction probability at the level of 70%, and 30% probability that will develop prediabetes.

5. The method according to claim 1, where the glycans under the peaks GP3, GP5, GP6, GP10, GP14, GP17, GP18, GP19, GP22, GP29, GP32, GP33, and GP36 are determined by alternative quantitative analytical techniques selected from the group consisting of: MALDI-TOF mass spectrometry, liquid chromatography coupled with mass spectrometry <LC-MS>, or capillary electrophoresis <CE>, or by LC-MS analysis of the corresponding glycopeptides.
